(19) United States
(12) Patent Application Publication
(10) Pub. No.: US 2025/0325817 A1
(43) Pub. Date: Oct. 23, 2025

(54) IMPLANTABLE CRANIAL NERVE STIMULATOR WITH RESPIRATION CYCLE DETECTION

(71) Applicant: Avivomed, Inc., Roseville, MN (US)

(72) Inventors: Paul Richard Mazanec, Ham Lake, MN (US); Stephen C. Masson, JR., Raleigh, NC (US)

(21) Appl. No.: 18/943,626

(22) Filed: Nov. 11, 2024

Related U.S. Application Data
(63) Continuation of application No. PCT/US2024/025682, filed on Apr. 22, 2024.

(51) Int. Cl. A61N 1/36; A61N 1/372; A61N 1/378

(52) U.S. Cl. CPC A61N 1/36139; A61N 1/3601; A61N 1/36053; A61N 1/36078; A61N 1/3611; A61N 1/37211; A61N 1/3787

(57) ABSTRACT

Neurostimulation therapy can be efficiently controlled based on information from an acceleration signal, such as can be obtained from an accelerometer. In an example, the accelerometer can be implanted in a cervical region or submandibular region of a patient. Circuitry can be configured to identify a first series of respiration phase transition events in the acceleration signal and, in response, provide the neurostimulation therapy synchronously with an inspiration phase of a patient's respiratory cycle. In an example, in absence of identifying the first series of respiration phase transition events in the acceleration signal, the neurostimulation therapy can be provided asynchronously with the patient's respiratory cycle.

### CROSS-REFERENCE TO RELATED APPLICATIONS

This patent application is a continuation of International Application No. PCT/US2024/025682, filed Apr. 22, 2024, the content of which is hereby incorporated by reference in its entirety

### BACKGROUND

Neural function can impact various disorders such as including cardiovascular disorders, movement disorders and tremors, epilepsy, depression, respiratory disorders (e.g., chronic obstructive pulmonary disease (COPD), pleural effusion), sleep disorders (e.g., obstructive sleep apnea (OSA)), obesity, xerostomia, and facial pain disorders. These disorders impact millions of patients and impact their quality of life and longevity. Obstructive sleep apnea, for example, is a common sleep disorder. Individuals suffering from OSA experience interrupted breathing patterns during sleep. Chronic, severe sleep apnea can require treatment to prevent sleep deprivation and other sleep-related complications. Obstructive sleep apnea is prevalent in patients with cardiovascular disease, is a cause of hypertension, and is associated with increased incidence of stroke, heart failure, atrial fibrillation, and coronary heart disease. Severe OSA is associated with an increase in all-cause and cardiovascular mortality.

In an example, external or implanted muscle stimulation devices or neurostimulation devices can be provided to excite tissue structures in or near an airway, such as to help treat sleep apnea or to counter apneic and hypopneic events.

In an example, neurostimulation can be used to treat a variety of disorders other than OSA. For example, neurostimulation can be used to treat epilepsy, depression, heart failure, obesity, pain, migraine headaches, COPD, or other disorders.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates generally a first anatomic example of front view of an anterior cervical region of a human.

FIG. 2 illustrates generally a second anatomic example that includes a portion of an anterior cervical triangle.

FIG. 3 illustrates generally a third anatomic example that includes a partial side view of an anterior cervical triangle.

FIG. 4 illustrates generally a fourth anatomic example that includes a partial side view of an anterior cervical triangle.

FIG. 5 illustrates generally a first implantable device implanted in a submandibular region of a patient.

FIG. 6 illustrates generally an example of a system that can be configured to provide a neuromodulation therapy.

FIG. 7 illustrates generally an example of an implantable neuromodulation system.

FIG. 8 illustrates generally an example of a physiologic parameter detection algorithm.

FIG. 9 illustrates generally an example of a respiration signal chart.

FIG. 10 illustrates generally an example of a first method that can include providing a neurostimulation therapy in coordination with an inspiration phase of a respiratory cycle of a patient.

FIG. 11 illustrates generally an example of a second method that can include determining a moving average for a physiologic sensor signal.

FIG. 12 illustrates generally an example of a third method that can include selectively providing a synchronous neurostimulation therapy or an asynchronous neurostimulation therapy.

FIG. 13 illustrates generally an example of a respiration signal chart.

FIG. 14 illustrates generally an example of a first method that can include providing a neurostimulation therapy in coordination with an inspiration phase of a respiratory cycle of a patient.

FIG. 15 illustrates generally an example of a machine in the form of a computer system within which a set of instructions may be executed for causing the machine to perform any one or more of the methodologies discussed herein, according to an example embodiment.

### DETAILED DESCRIPTION

Systems, devices, and methods discussed herein can be configured for electrical stimulation of cranial nerves. Examples discussed herein can include methods for implanting a neuromodulation system or methods for using an implanted system to deliver neuromodulation therapy to one or more target cranial nerves, or to sense physiologic information about a patient, such as to monitor a disease state or control a neuromodulation therapy or other therapy. In an example, system or device features discussed herein can augment devices, leads, sensors, electrostimulation hardware, or other therapeutic means at, on, or near cranial nerve tissue. In an example, the present subject matter includes systems and methods for using a neuromodulation device that is implanted near or below an inferior border of a mandible (i.e., the body or ramus of the mandible or jaw bone) in an anterior triangle of the neck (e.g., located in the medial aspect), or in a posterior triangle of the neck (e.g., located in the lateral aspect), or in other cervical regions.

The present inventors have recognized that a problem to be solved can include providing a minimally invasive neuromodulation therapy or treatment system that can provide signals to neural targets in or near a cervical region of a patient. The problem can include treating, among other things, obstructive sleep apnea (OSA), heart failure, hypertension, epilepsy, depression, post-traumatic stress disorder (PTSD), attention deficit hyperactivity disorder (ADHD), craniofacial pain syndrome, facial palsy, migraine headaches, xerostomia, atrial fibrillation, stroke, autism, inflammatory bowel disease, chronic inflammation, chronic pain, tinnitus, rheumatoid arthritis, hyperthyroidism, hypothyroidism, certain cancers, or fibromyalgia. The problem can include providing an implantable system that can chronically detect a patient respiratory status or respiratory cycle with minimal power consumption and improved accuracy, to enhance an efficacy of an apnea treatment.

The present inventors have recognized, among other things, that a solution to the above-described problems can include a neuromodulation system that can be implanted in an anterior cervical region of a patient, such as at or under a mandible of the patient, or in a submandibular region. In an example, the system can include a housing that can be coupled to tissue in or near an anterior triangle, such as can be coupled to digastric muscle or tendon tissue, to mylohyoid muscle tissue, to a hyoid bone, or to a mandible, among other locations. The present inventors have recognized that the solution can include or use a sensor, such as an accelerometer, implanted with the system in the anterior cervical region and configured to sense information about tongue movement, motion, force, pressure, electrical activity, bioimpedance, or other information that can indicate tongue muscle behavior. In an example, the accelerometer can be configured to detect motion or acoustic information that includes information about an upper airway air flow or breath (e.g., respiration). In an example, the accelerometer can be configured to sense a response to a stimulation therapy provided by the system.

The present inventors have recognized that the neuromodulation systems and methods discussed herein can be used to treat OSA, among other disorders or diseases. In an example, an OSA treatment can use a neuromodulation device that is implanted in a cervical region, such as can include a submandibular region. The cervical region can include one or more of a submental triangle and a submandibular triangle region. In an example, the neuromodulation system can comprise an electrode lead with one or more electrodes that are configured to be disposed at or near one or more targets on a hypoglossal nerve, vagus nerve, glossopharyngeal nerve, ansa cervicalis, or trigeminal nerve (e.g., at a mandibular branch of the trigeminal nerve). In an example, the solution can include using multiple electrodes or electrode leads to deliver a coordinated stimulation therapy to one or multiple cranial nerve targets. For example, the therapy can include bilateral stimulation of branches of the hypoglossal nerve, or stimulation of multiple different nerves. The therapy can be configured to selectively stimulate or block a neural pathway that influences activity of one or more of tongue muscles, mylohyoid muscles, stylohyoid muscles, digastric muscles, or stylopharyngeus muscles of a patient, to thereby treat OSA or other conditions.

The description that follows describes systems, methods, techniques, instruction sequences, and computing machine program products that illustrate example embodiments of the present subject matter. In the following description, for purposes of explanation, numerous specific examples and aspects are set forth in order to provide an understanding of various embodiments of the present subject matter. It will be evident, however, to those skilled in the art, that embodiments of the present subject matter may be practiced in various combinations. Unless explicitly stated otherwise, structures (e.g., structural components, such as modules or functional blocks) are optional and may be combined or subdivided, and operations (e.g., in a procedure, algorithm, treatment, therapy, or other function) can vary in sequence or can be combined or divided.

In an example, the implantable neuromodulation systems and devices discussed herein can comprise a control system, signal or pulse generator, or other therapy signal generator, such as can be disposed in one or more housings that can be communicatively coupled to share power and/or data. The housings can comprise one or more hermetic enclosures to protect the circuitry or other components therein. In an example, a housing can include one or more headers, such as can comprise a rigid or flexible interface for connecting the housing, or circuitry or components inside of the housing, with leads or other devices or components outside of the housing. In an example, a header can be used to couple signal generator circuitry inside the housing with electrodes or sensors outside of the housing. In some examples, the header can house one or more sensors. In an example, the header can be used to couple circuitry inside the housing with a telemetry antenna, wireless power communication devices (e.g., coils configured for near-field communications or NFC), or other devices, such as can be contained within the header or disposed on or comprise flexible substrates or flexible circuits. This system configuration allows the housing(s), lead(s), and flexible circuits to be implanted in different anatomic locations, such as in a neck or cervical region of a patient. In an example, the various system components can be implanted in one or more of the anatomic triangular regions or spaces in the cervical region, and leads or other devices external to a circuitry housing can be tunneled to other locations, including at various cranial nerve targets. Accordingly, various therapeutic elements can be implanted on or near target cranial nerves, and sensing elements can be implanted on or near the same or other cranial nerves or at other anatomic structures in the same or different locations. Some components can be located in a different anatomic location, such as in a different cervical region than is occupied by a housing. For example, a telemetry antenna or NFC coil can be provided at or near a surface of the skin, while a housing with circuitry that coordinates neuromodulation therapy or power signal management can be implanted elsewhere, such as more deeply within one or more cervical regions.

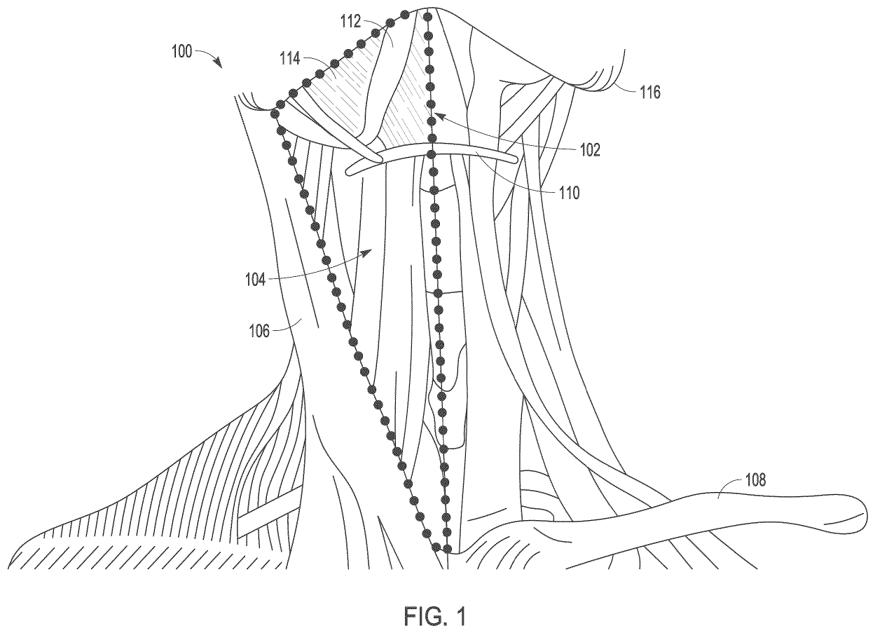

FIG. 1 illustrates generally a first anatomic example 100 of a front view of an anterior cervical region of a human. The region generally extends between a clavicle 108 and mandible 116 and can be divided into various additional regions or subregions. In an example, the anterior cervical region includes a pair of anterior triangles on opposite sides of a sagittal midline 102, such as including an anterior triangle 104 as illustrated. The term “midline” as used herein refers to a line or plane of bilateral symmetry in the cervical or neck region of a person. In an example, a midline corresponds to the sagittal plane, that is, is the anteroposterior (AP) plane of the body.

The anterior triangle 104 can include a region that is bounded by the midline 102, a base of the mandible 116, and a sternocleidomastoid muscle, or SCM 106. A hyoid bone 110 can extend between the pair of anterior triangles across the midline 102. The anterior triangle 104 can include, among other things, a digastric muscle 112 (e.g., including anterior and posterior portions of the digastric muscle 112), a mylohyoid muscle 114, and various other muscle, bone, nerve, and other body tissue.

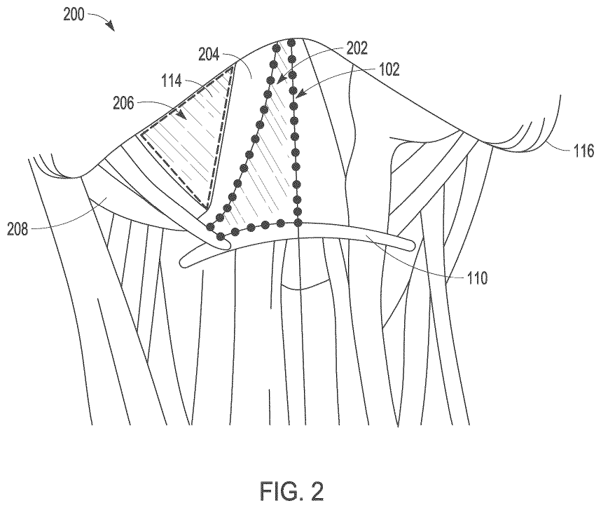

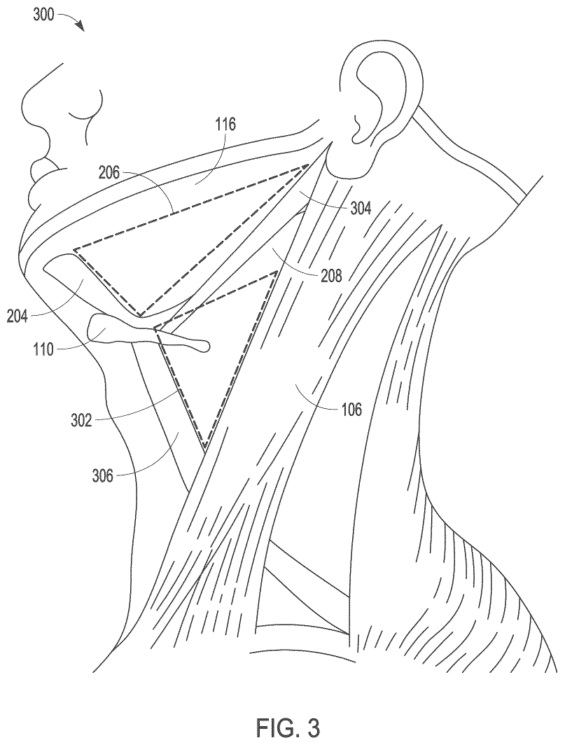

FIG. 2 illustrates generally a second anatomic example 200 that includes a portion of the anterior triangle 104 from the example of FIG. 1. FIG. 2 shows, for example, that the anterior triangle 104 can be divided into various regions, including a submandibular triangle 206 and a submental triangle 202. In an example, the anterior triangle 104 can include a carotid triangle, as discussed below in the example of FIG. 3. A posterior triangle of the neck (not shown) can be divided into various regions, including an occipital triangle and a supraclavicular triangle.

The submental triangle 202 is generally understood to include a region that is bounded by the midline 102, the hyoid bone 110, and the anterior digastric muscle 204. The submandibular triangle 206 is generally understood to include a region that is bounded by the anterior digastric muscle 204, the posterior digastric muscle 208, and a base of the mandible 116.

FIG. 3 illustrates generally a third anatomic example 300 that includes a partial side view of the anterior triangle 104. The example of FIG. 3 further illustrates the location of the submandibular triangle 206, such as in relation to the anterior digastric muscle 204 and the mandible 116. The example of FIG. 3 illustrates the carotid triangle 302, such as can comprise a portion of the anterior triangle 104 in the cervical region. The carotid triangle 302 is generally understood to include a region that is bounded by the SCM 106, the omohyoid muscle 306, and the posterior digastric muscle 208.

In an example, an implantable neuromodulation device can be implanted in the anterior triangle 104 or in the posterior triangle, such as using the systems and methods discussed herein. In further examples, an implantable neuromodulation device can be implanted in one or more of the submental triangle 202 and the submandibular triangle 206. The implantable neuromodulation device can be configured to provide a stimulation therapy to one or multiple nerve targets such as can be in or near the anterior triangle 104 or the posterior triangle, or to nerve targets that can be accessed via tunneled leads that extend from a housing that is disposed in a cervical region, such as in the anterior triangle 104 or the posterior triangle. In other words, various regions in the anterior and posterior cervical triangles can provide access to a main body of, or to branches of, various cranial nerves, including the hypoglossal nerve (CN XII), the accessory nerve (CN XI), the vagus nerve (CN X), the glossopharyngeal nerve (CN IX), the facial nerve (CN VII), and the trigeminal nerve (CN V), among others.

The present inventors have realized that the anterior and posterior cervical triangles are anatomic locations suitable for implantation of a neuromodulation system or component thereof. The present inventors have further realized that the locations include various anatomic structures suitable for coupling and therefore stabilizing a neuromodulation system or component thereof. For example, the present inventors have recognized that such coupling structures can include the hyoid bone 110, the connective tissue sling of the hyoid bone 110, the mandible 116, the digastric tendon, the anterior or posterior portion of the digastric muscle 112, the stylohyoid muscle 304, the mylohyoid muscle 114, the omohyoid muscle, or the SCM 106. The present inventors have recognized that the submental triangular region is suitable for implantation of a neuromodulation system. The submental triangular region is generally bounded superiorly by the mylohyoid and inferiorly by the digastric muscle. By implanting the system inferior to the mylohyoid and between the digastric muscles, a minimally invasive procedure can be used.

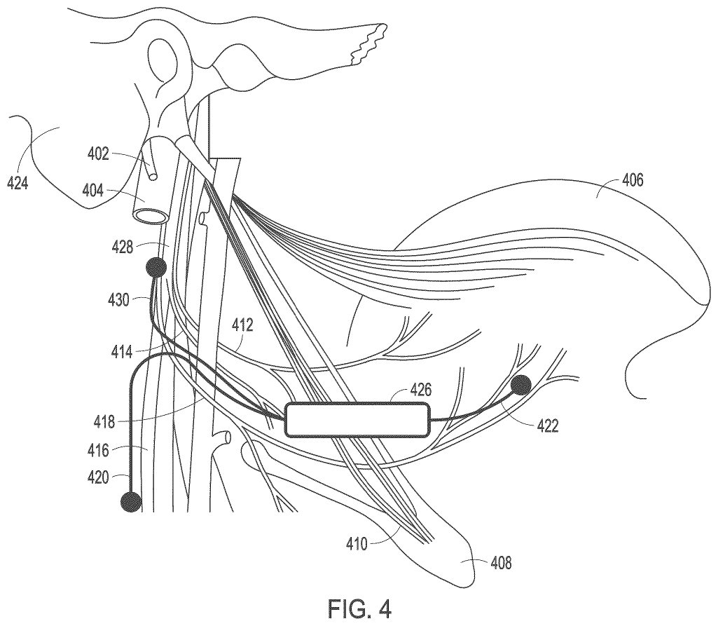

FIG. 4 illustrates generally a fourth anatomic example 400 that includes a partial side view that includes the anterior triangle 104 of the cervical region. The fourth anatomic example 400 illustrates an upper portion of the anterior triangle 104 and a portion of the upper neck, such as at or below a temporal bone 424. A representation of a tongue 406 and of a portion of a jugular vein 404 is included for further context and reference.

The fourth anatomic example 400 shows various nerves and vessels. The illustrated nerves include some, but not all, of the cranial nerves that can be targeted using the neuromodulation systems, devices, and methods discussed herein. For example, nerve targets in the fourth anatomic example 400 include a facial nerve 402, a jugular vein 404, a glossopharyngeal nerve 412, a pharyngeal branch of vagus nerve 414, a vagus nerve 416, a hypoglossal nerve 418, and a mandibular branch of the trigeminal nerve 428, among others.

The example of FIG. 4 includes an example of an implantable therapy device 426. The implantable therapy device 426 can be implanted in a patient in an upper portion of an anterior triangle 104 of a cervical region of the patient. For example, the implantable therapy device 426 can be implanted in one or more of the submental triangle 202 and the submandibular triangle 206. In the example of FIG. 4, the implantable therapy device 426 can be coupled to various anatomical structures, such as a stylohyoid muscle 410, a hyoid bone 408, or other tendons or structures in the upper neck.

The example of FIG. 4 includes multiple leads coupled to the implantable therapy device 426. For example, the implantable therapy device 426 can be coupled to a lower electrode lead 420, an anterior electrode lead 422, and an upper electrode lead 430. The lower electrode lead 420 can be implanted at or near a neural target on the vagus nerve 416, for example, in or adjacent to the carotid triangle 302. In an example, the lower electrode lead 420 can be coupled to the SCM 106 or other structure at or near the vagus nerve 416. The upper electrode lead 430 can be implanted at or near the facial nerve 402, the mandibular branch of the trigeminal nerve 428, or the glossopharyngeal nerve 412, among others. In an example, the anterior electrode lead 422 can be implanted at or near a neural target on the hypoglossal nerve 418.

In an example, the various implantable devices and components thereof that are discussed herein can be coupled to various anatomic structures or tissues inside a patient body, such to stabilize or maintain a device or component at a particular location and resist device movement or migration as the patient carries out their daily activities. In an example, coupling a device or component to tissue can include anchoring, affixing, attaching, or otherwise securing the device or component to tissue using a coupling feature. A coupling feature can include, but is not limited to, a flap or flange, such as for suturing to tissue (e.g., muscle, tendon, cartilage, bone, or other tissue).

In an example, a coupling feature can include various hardware such as a screw or helical member that can be driven into or attached to tissue or bone. In an example, a coupling feature can include a cuff, sleeve, adhesive, or other component. In an example, one or multiple different coupling features can be used for different portions of the same neuromodulation system. For example, a suture can be used to couple a device housing to a tissue site, and a lead, such as coupled to the housing, can include tines or a distal cuff to secure the lead at or near a neural target.

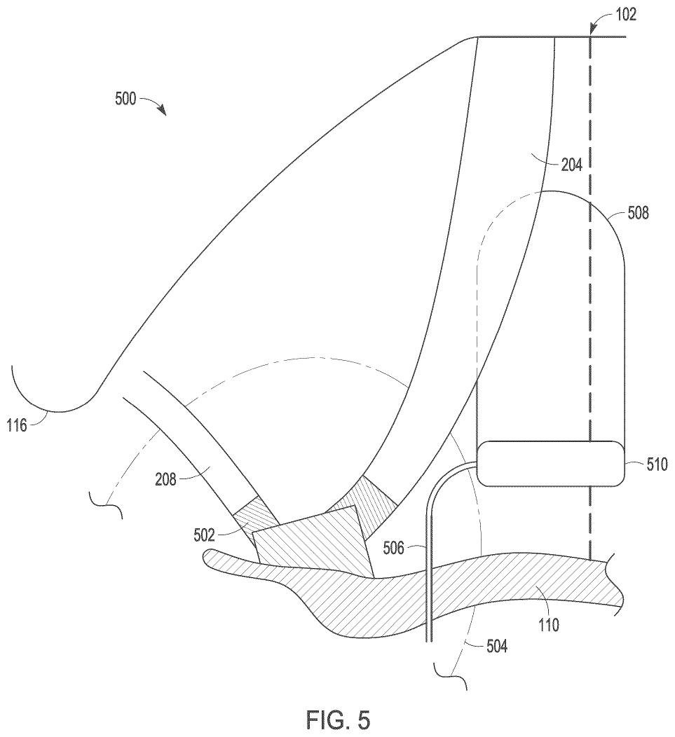

FIG. 5 illustrates generally a first example 500 that includes a first implantable device 508 implanted in the submental triangle 202 of a patient. In the first example 500, the first implantable device 508 can be coupled to an anatomic structure such as using a suture, anchor, or other affixation means. In an example, the first implantable device 508 can be coupled to one or more of the mandible 116, the anterior digastric muscle 204, the mylohyoid muscle 114, the digastric tendon 502, or other bone, tendon, muscle, or other structure that is in or adjacent to the submental triangle 202. In the example of FIG. 5, the first implantable device 508 can be provided near, but spaced apart from, a submandibular gland 504 of the patient. In an example, the first implantable device 508 is implanted and installed such that at least a portion of the device is disposed over the midline 102. That is, respective portions of the first implantable device 508 can be located on opposite sides of the midline 102. In an example, a central axis of the housing of the first implantable device 508 can be aligned with the midline 102.

In the example of FIG. 5, the first implantable device 508 includes a first header 510. The first header 510 can be used to couple one or multiple electrode leads, sensor leads, or other devices to the first implantable device 508. For example, the first header 510 can be used to couple the first implantable device 508 to a first electrode lead 506, and the first electrode lead 506 can be tunneled to a cranial nerve target. Electrodes configured to deliver electrostimulation signals to the nerve target can be situated at or adjacent to the target. In an example, the first electrode lead 506 can be tunneled to a hypoglossal nerve in or near the cervical region of a patient.

In the example of FIG. 5, the first implantable device 508 is shown with one header. The first implantable device 508 can optionally include multiple headers to interface the first implantable device 508 with one or multiple other leads, such as electrode leads, sensor leads, communication coils, or other devices. Referring again to FIG. 4, for example, the implantable therapy device 426 can include multiple headers, such as coupled to the respective different leads that extend from opposite sides of a body of the implantable therapy device 426.

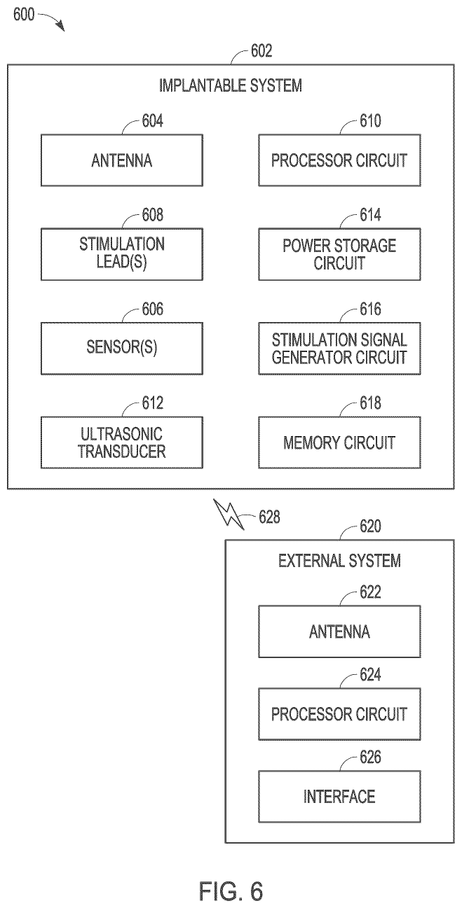

FIG. 6 illustrates generally an example of a system 600 that can be configured to provide or control a neuromodulation therapy. The system 600 can include an implantable system 602 and an external system 620. The implantable system 602 and the external system 620 can be communicatively coupled using a wireless coupling 628. In an example, the wireless coupling 628 can enable power signal communication (e.g., unidirectionally from the external system 620 to the implantable system 602), or can enable data signal communication (e.g., bidirectionally between the implantable system 602 and the external system 620). In an example, the implantable system 602 or the external system 620 can be wirelessly coupled for power or data communications with one or more other devices, including other implantable or implanted devices, such as in the same patient body.

In the example of FIG. 6, the implantable system 602 can include an antenna 604, a sensor(s) 606 such as comprising one or more physiologic sensors, a stimulation lead(s) 608, a processor circuit 610, an ultrasonic transducer 612, a power storage circuit 614, a stimulation signal generator circuit 616, and a memory circuit 618, among other components or modules.

In an example, the antenna 604 can include a telemetry antenna such as configured for data communication between the implantable system 602 and the external system 620. In an example, the antenna 604 can include an antenna, such as an NFC coil, that is configured for wireless power communication between the implantable system 602 and the external system 620 or other external power source. In some examples, the same antenna 604 is configured for concurrent power and data communication.

The processor circuit 610 can include a general purpose or purpose-built processor. The memory circuit 618 can include a long-term or short-term memory circuit, such as can include instructions executable by the processor circuit 610 to carry out therapy or physiologic monitoring activities for the system 600. In an example, the processor circuit 610 of the implantable system 602 is configured to manage telemetry or data signal communications with the external system 620, such as using the antenna 604 or other communication circuitry. In an example, the processor circuit 610 is configured to execute one or more algorithms that are configured to use physiologic signal information to identify a respiration cycle of a patient. The algorithms can be configured to provide information about respiration cycle phases, or phase transitions, such that an inspiration phase or an exhalation phase can be identified. In an example, the algorithms can include pattern matching or signal comparison functions, such as can be leveraged to identify respiration cycle information.

In an example, the stimulation signal generator circuit 616 includes an oscillator, pulse generator, or other circuitry configured to generate electrical signals that can provide electrostimulation signals to a patient body, or to power various sensors (e.g., including the sensor(s) 606), or transducers (e.g., including the ultrasonic transducer 612). In an example, the stimulation signal generator circuit 616 can be configured to generate multiple electrical signals to provide multipolar electrostimulation therapy to multiple neural targets, such as concurrently or in a time-multiplexed manner. The stimulation signal generator circuit 616 can be configured to use or provide different neurostimulation signals, such as can have different pulse amplitude, pulse duration, waveform, stimulation frequency, or burst pattern characteristics.

The stimulation signal generator circuit 616 can be used to generate therapy signals for multiple different targets concurrently. For example, signals from the stimulation signal generator circuit 616 can be used to stimulate one cranial nerve target to efferent effect, and to stimulate a different nerve or branch to elicit an afferent response. In another example, one cranial nerve can be blocked while another nerve is stimulated. Other combinations can similarly be used.

In an example, the processor circuit 610 is configured to control the stimulation signal generator circuit 616. That is, the stimulation signal generator circuit 616 can generate stimulation signals in response to control signals from the processor circuit 610. For example, the processor circuit 610 can coordinate generation and delivery of the stimulation signals based on physiologic status information, such as respiratory cycle information, such as can be determined by the processor circuit 610 using information from the sensor(s) 606.

In an example, the stimulation lead(s) 608 can include one or more leads that are coupled to or integrated with a housing or header of the implantable system 602. The stimulation lead(s) 608 can be detachable from the housing to facilitate replacement or repair.

In an example, the stimulation lead(s) 608 can include electrostimulation hardware such as electrodes having various configurations, including cuff electrodes, flat electrodes, percutaneous electrodes or other configurations suitable for electrical stimulation of nerves or nerve bodies or branches. In an example, the stimulation lead(s) 608 can additionally or alternatively comprise other neuromodulation therapy hardware such as the ultrasonic transducer 612, drug delivery means, or a mechanical actuator, such as can be configured to modulate neural activity. The stimulation lead(s) 608 can include one or more electrodes that are configured to sense electrical activity from a patient body. For example, one or more of the electrodes can be configured to monitor an electrical response from nerve or muscle tissue of the patient body. In an example, the one or more electrodes of the stimulation lead(s) 608 can be used to receive information about an evoked compound action potential, or ECAP, such as can indicate a type or amount of neural fiber that is activated in response to a stimulation. In some examples, the stimulation can be provided using one or more of the same electrodes in the stimulation lead(s) 608 as used to receive the ECAP information, or the stimulation can be provided using other electrodes. The processor circuit 610 can be configured to receive the information about the ECAP and identify characteristics of the evoked response, such as can be used to assess an effectiveness of a neuromodulation therapy.

The leads and/or electrodes discussed herein can have various features that can facilitate placement at, and stimulation of, one or more neural targets. A lead can have one or more electrodes that can be used for nerve stimulation, nerve blocking, or nerve sensing. The electrodes can have various surface area and spacing characteristics (e.g., spacing from other electrodes, sensors, targets, etc.) to optimize for a particular function. In an example, an electrode can comprise various materials, including low-oxidation metals or metal alloys (e.g., platinum, platinum iridium, etc.) for use in implantable systems. In an example, an electrode can be treated or coated with another material such as to promote healing or enhance charge transfer to tissue.

In an example, an electrode lead can comprise one or multiple electrodes, such as can have the same or different electrode characteristics. A lead can include, for example, a spiral electrode or cuff electrode. In such an example, one or more conductive surfaces can be exposed on an inside surface of a curved or spiral cuff assembly such as can comprise a portion of a lead body. In an example, a spiral cuff assembly (and hence, electrodes) can be designed to circumferentially wrap snugly around a body of a nerve and can be self-sizing. In an example, a cuff electrode can be configured to surround a particular target to thereby direct stimulation energy to the target from multiple different directions concurrently, such as while insulating the electrode from adjacent tissue.

In an example, a percutaneous electrode can be used, such as including one or more electrodes exposed on a lead that is inserted into a blood vessel (or other conducting tissue in the vicinity of a neural target) using percutaneous techniques. A percutaneous lead can be navigated by a clinician, within or through vasculature, toward target nerves or neural structures that are in close proximity to the vasculature. In an example, electrodes on a percutaneous lead can be directly on the lead body or can comprise a percutaneous structure, such as a stent-like frame or scaffold, whereby the electrodes can be oriented towards the target and away from the blood in the vessel.

In an example, a bifurcated lead can be used to provide electrodes at multiple different and spaced apart anatomical targets while using a single connection to a header. In an example, a modular lead can be used such as to extend or tailor a lead to accommodate a patient's anatomy or target structures. In some examples, a housing of the various devices discussed herein can include one or more electrodes configured for use in electrostimulation delivery. Each of the electrodes in or coupled to the implantable system 602 can be separately addressable by neuromodulation therapy control or coordination circuitry (e.g., the processor circuit 610) to deliver a coordinated therapy to one or multiple targets, or to sense a response (e.g., an ECAP response, an acceleration signal indicative of a muscle response, etc.) at one or multiple locations.

Various stimulation configurations can be used with any of the electrode or lead types discussed herein. In an example, different configurations can be used to provide or modify a stimulating electric field to thereby affect an extent and manner of neural excitation. The configurations can include, for example, unipolar, bipolar, and various combinations of multipolar configurations. In a bipolar or multipolar configuration, a guard electrode can be used to help steer excitation or inhibit neural activity. In an example, an electrode configuration can be dynamically changed, such as throughout the course of a particular therapy, such as through programming changes or during operation to achieve a particular therapy.

In an example, the sensor(s) 606 can include, among other things, electrodes for sensing of electrical activity such as using electrocardiograms (ECGs), impedance, electromyograms (EMGs) of select muscles, electroencephalograms (EEGs), and/or electroneurograms (ENGs) of target cranial nerves and branches. The sensor(s) 606 can include pressure sensors, photoplethysmography (PPG) sensors, chemical sensors (e.g., pH, lactate, glucose, etc.) or other sensors that can be used for physiologic sensing of cardiac, respiratory, or other physiologic activity.

In an example, the sensor(s) 606 can include an accelerometer (e.g., configured to sense acceleration information along one or multiple axes), gyroscope or geomagnetic sensor, such as can be configured to measure patient or device movement, vibration, position, posture, or other orientation information. Other examples of the sensor(s) 606 are discussed elsewhere herein, including in the discussion of the machine 1500 and the various I/O components 1542, such as including the biometric components 1532, motion components 1534, and environmental components 1536. In an example, information from the sensor(s) 606 can be received by the processor circuit 610 and used to update or titrate a neuromodulation therapy.

In an example, the implantable system 602 can include one or more sensor(s) 606, such as can be used in providing closed-loop neuromodulation therapy that is based at least in part on physiologic status information about a patient (e.g., respiration, heart rate, blood pressure, neural or muscular activation, or other information). In an example, the sensor(s) 606 can be used to receive diagnostic information, or to receive information about patient movement or body position or posture.

In an example, hypoglossal nerve stimulation, such as to treat OSA, can be controlled at least in part based on information from an accelerometer or gyroscope to determine patient respiration, patient activity, and body orientation or position, such as together with information from a pressure sensor about respiration. In other words, using information from the sensor(s) 606, such as including accelerometer and pressure sensors, the implantable system 602 can control neuromodulation therapy provided to the hypoglossal nerve, such as can include stimulation during a particular time within a respiratory cycle, and can use body position information to automatically enable therapy when, for example, the patient is sleeping.

In an example, information from multiple different sensors can be used together to cross-check or validate physiologic status information, or to help improve immunity from noise or other aberrations in sensor data. In some examples, primary and second sensors can be used together, and information from the secondary sensor can be used in the event of a primary sensor failure or unavailability.

In an example, acceleration information from multiple different axes, such as from the same or different accelerometer, can be used to identify patient posture or respiration status. The present inventors have found, for example, that acceleration information from each of multiple axes, such as from the same multiple-axis accelerometer that is implanted in a cervical region, such as the submental region, can be used together to more accurately determine respiration cycle information about the patient. For example, acceleration information from a first axis may provide relatively high signal to noise for respiration cycle information when a patient is in a first posture, but may provide relatively low signal to noise for respiration cycle information when a patient is in a different second posture. The processor circuit 610 can be configured to identify and prioritize the acceleration data with the highest signal to noise ratio for the particular physiologic status information of interest. In some examples, acceleration information from multiple axes can be received and used together as a composite acceleration signal. In some examples, acceleration information from one or more axes can be pre-processed or filtered to help best identify physiologic status information of interest.

In an example, the sensor(s) 606 includes an accelerometer positioned to capture the nuanced movements associated with a patient's respiratory cycle. The processor circuit 610 can be configured to process data received from the accelerometer and employ algorithms to determine a jerk signal, which represents a rate of change of acceleration over time. The jerk signal can be used to identify shifts that signify transitions between the inhalation and exhalation phases of the respiratory cycle. By analyzing the jerk signal, the processor circuit 610 can accurately pinpoint the timing of these respiratory phases.

In an example, the processor circuit 610 or the external system 620 can be configured to use information about a therapy to receive or interpret data from the sensor(s) 606. For example, some sensor information may be corrupted or otherwise influenced by an electrostimulation therapy provided by the implantable system 602 or by another therapy or event provided by the same or other implanted or external device. In some examples, sensor information can be “blanked” or disregarded at, during, or for a specified period following a stimulation therapy delivery event.

In the example of FIG. 6, the external system 620 can include various components that can be provided together as a unitary external device or can include multiple devices configured to work together to manage a patient therapy, manage a device such as the implantable system 602, or perform other functions associated with the implantable system 602. The external system 620 can include an antenna 622, a processor circuit 624, and an interface 626, among other components or modules.

The antenna 622 can comprise one or multiple antennas such as can be configured for nearfield or farfield communications with, for example, the antenna 604 of the implantable system 602, a different implantable device or system, or other external device. In an example, the antenna 622 and the antenna 604 can be used to exchange power or data between the implantable system 602 and the external system 620. For example, information about a prescribed therapy can be uploaded from the external system 620 to the implantable system 602, or information about a physiologic status, such as measured by the sensor(s) 606, can be downloaded from the implantable system 602 to the external system 620.

The processor circuit 624 can include a general purpose or purpose-built processor configured to carry out various activities on the external system 620 or in coordination with the implantable system 602. In an example, the processor circuit 624 of the external system 620 is configured to manage telemetry or data signal communications with the implantable system 602, such as using the antenna 622 or other communication circuitry.

The interface 626 can include a patient or clinician interface, such as to report device information or to receive instructions or therapy parameters for implementation by the implantable system 602. In an example, the interface 626 can include an interface or gateway to facilitate communication between the 602 or the external system 620 with a patient management system or other medical record system. Other features, modules, and components of the implantable system 602 and the external system 620 can be included in the system 600 to help administer various neuromodulation therapies.

In an example, the systems, devices, and components discussed herein, including at least the implantable system 602 and the external system 620 of the system 600, can be used to provide neuromodulation therapy to nerve targets inside a patient body, such as to treat one or more disorders or diseases. In an example, the system 600 or components thereof can be configured to provide neuromodulation therapy to multiple nerve targets in a coordinated manner, such as concurrently, or in a time-multiplexed sequence. In an example, the neuromodulation therapy can include one or more, or combinations of, neural stimulation and blocking signals, such as can be directed to afferent or efferent nerve structures or targets to trigger different responses. The therapy can optionally include using vector-based stimulation configurations to target particular nerves or nerve regions, or can include more relatively targeted or isolated nerve fibers. In an example, a coordinated neuromodulation therapy can include blocking at a first nerve target, while stimulating a second nerve target, or concurrently (or in time-sequence) stimulating multiple different nerve targets.

In an example, the particular patient disorder or disease can dictate the particular neural target to modulate with a neuromodulation therapy. For example, to treat obstructive sleep apnea using the system 600, various cranial nerves can be targeted individually or together, such as including the trigeminal nerve (e.g., the V3 mandibular branch of the trigeminal nerve 428), the hypoglossal nerve 418 (e.g., including one or more branches thereof), the glossopharyngeal nerve 412, the vagus nerve 416, or the facial nerve 402 (eg., including various extracranial branches thereof).

In an example, the system 600 can be used to treat OSA by providing a neuromodulation therapy to or including the mandibular branch of the trigeminal nerve 428 and the hypoglossal nerve 418. In this example, neuromodulation of the mandibular branch of the trigeminal nerve 428 can influence motor control of the mylohyoid muscle 114 or the anterior digastric muscle 204, and neuromodulation of the hypoglossal nerve 418 can influence motor control of muscles in the tongue 406.

In an example, the system 600 can be used to treat OSA by providing a neuromodulation therapy to or including the facial nerve 402 and to the hypoglossal nerve 418. In this example, neuromodulation of the facial nerve 402 can influence motor control of the stylohyoid muscle 304 or the posterior digastric muscle 208, and neuromodulation of the hypoglossal nerve 418 can influence motor control of muscles in the tongue 406.

In an example, the system 600 can be used to treat OSA by providing a neuromodulation therapy to or including the glossopharyngeal nerve 412 and the hypoglossal nerve 418. In this example, neuromodulation of the glossopharyngeal nerve 412 can influence motor control of the stylophryngeus muscle, and neuromodulation of the hypoglossal nerve 418 can influence motor control of muscles in the tongue 406.

In an example, the system 600 can be used to treat OSA by providing a neuromodulation therapy to or including various branches of the hypoglossal nerve 418, including anterior branches, posterior branches, or multiple branches concurrently, including or using a bilateral configuration to target branches on opposite sides of the midline 102 of a patient. The neuromodulation of the hypoglossal nerve 418 can influence motor control of various muscles in the tongue 406. In an example, neuromodulation therapy that includes stimulating or blocking the hypoglossal nerve 418 can be combined with therapy that targets one or more of the mandibular branch of the trigeminal nerve 428 (e.g., to influence motor control of the mylohyoid muscle 114 or the anterior digastric muscle 204), the facial nerve 402 (e.g., to influence motor control of the stylohyoid muscle 304 or the posterior digastric muscle 208), or the glossopharyngeal nerve 412 (e.g., to influence motor control of the stylophryngeus muscle), among others.

Any one or more branches of the hypoglossal nerve 418 can receive a neuromodulation therapy from the implantable system 602. For example, any one or more of the posterior branches of the hypoglossal nerve 418 can receive neuromodulation, including for example “branches” off the hypoglossal nerve sheath such as the meningeal branch (B1), the vascular branch (B2), the descending branch, also referred to as the superior root of the ansa cervacalis (B3), the thyrohyoid branch (B4), or the geniohyoid branch (B5). Any one or more of the anterior branches of the hypoglossal nerve 418 can receive neuromodulation, including for example where a main trunk of the hypoglossal nerve 418 branches to the muscles of the tongue, also referred to as the muscular branch (B6), or including the muscular branch itself. The muscular branch can include sub-branches or nerve fibers that innervate specific muscles of the tongue.

In an example, the system 600 can be used to treat OSA or other disorders or diseases such as heart failure, hypertension, atrial fibrillation, epilepsy, depression, stroke, autism, inflammatory bowel disease, chronic inflammation, chronic pain (e.g., in cervical regions, in the lower back, or elsewhere), tinnitus, or rheumatoid arthritis, cancer, or thyroid disorders, among others, such as by providing a neuromodulation therapy to or including the vagus nerve 416. Neuromodulation of the vagus nerve 416 can influence parasympathetic tone to thereby treat or alleviate symptoms associated with the various diseases or disorders mentioned, among others. In an example, a therapy that includes stimulation of the vagus nerve 416 can include therapy provided to one or more branches of the hypoglossal nerve 418, the mandibular branch of the trigeminal nerve 428, the facial nerve 402, or the glossopharyngeal nerve 412. In an example, neuromodulation therapy that includes stimulating or blocking a portion of the vagus nerve 416 can be combined with therapy that targets one or more of the glossopharyngeal nerve 412 (e.g., to further influence parasympathetic tone), the carotid sinus (e.g., to stimulate a baroreceptor response), or the superior cervical ganglion or branches thereof (e.g., to influence sympathetic tone).

In an example, a neuromodulation therapy for treatment of heart failure, hypertension, and/or atrial fibrillation can include therapy provided to or including one or more of the glossopharyngeal nerve 412 (e.g., to influence parasympathetic tone, such as via communication to the vagus nerve 416), the superior cervical ganglion (e.g., to influence sympathetic tone), or the carotid sinus (e.g., to stimulate a baroreceptor response).

In an example, the system 600 can be configured to treat heart failure, hypertension, migraine headaches, xerostomia, or other diseases or disorders by providing a neuromodulation therapy to or including the glossopharyngeal nerve 412. Stimulation or blocking of the glossopharyngeal nerve 412 can, for example, influence parasympathetic tone or can affect motor activity of the stylopharyngeus muscle.

In an example, the system 600 can be configured to treat drug-refractory epilepsy, depression, post-traumatic stress disorder (PTSD), migraine headaches, attention-deficit hyperactivity disorder (ADHD), craniofacial pain syndrome, among other diseases and disorders, such as by providing a neuromodulation therapy to or including the mandibular branch of the trigeminal nerve 428.

In an example, the system 600 can be configured to treat craniofacial pain syndrome, or facial palsy, among other things, such as by providing a neuromodulation therapy to or including the facial nerve 402, such as including various extracranial branches or roots thereof. In an example, the system 600 can be configured to treat fibromyalgia such as by providing a neuromodulation therapy to or including the spinal accessory nerve, such as to target the trapezius muscle, which is understood to be a potential trigger point for fibromyalgia. In an example, the system 600 can be configured to treat migraine headaches or tinnitus, such as by providing a neuromodulation therapy to or including a great occipital nerve, such as can be accessed using electrodes implanted in the cervical region of a patient.

Neuromodulation therapies can thus be provided using the system 600, or using components thereof, to treat a variety of different diseases or disorders. The therapies can include targeted, single-location stimulation or blocking (e.g., using electrical pulses, ultrasonic signals, or other energy) therapy at one of the locations mentioned herein (among others) or can include coordinated stimulation or blocking across or using multiple different locations. The following discussion illustrates several examples of different implantation locations and neural targets, however, others including those specifically mentioned above, can similarly be used.

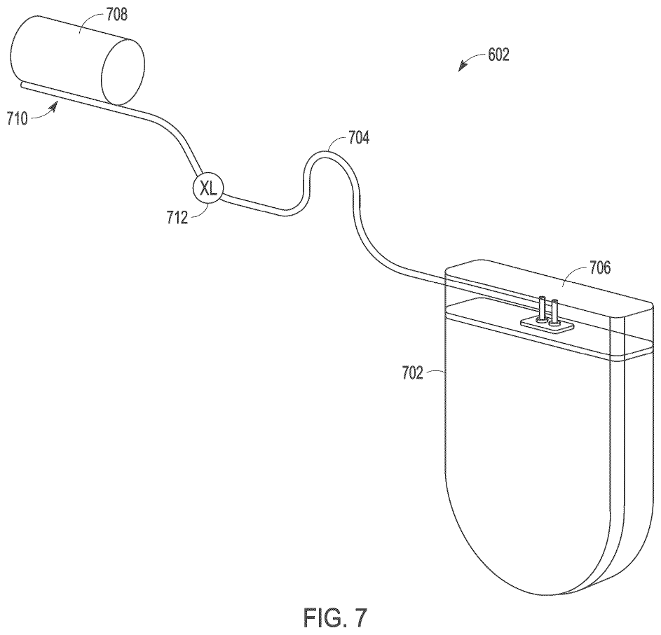

In an example, the implantable system 602 comprises an implantable housing (sometimes referred to as a “can”) or body portion that includes a flattened or compressed half-capsule (or other portion of a capsule) structure, as shown in FIG. 7. The housing can extend along a longitudinal axis and includes rounded ends or caps. In an example, the housing is a conductive housing 702 that can be configured as a reference electrode for use in electrostimulation delivery or electrical response sensing.

The implantable body can include a header 706 for interfacing with one or multiple leads. In the example of FIG. 7, the implantable body can be coupled to a single lead 704 having a distal cuff 708 that includes an electrode array 710. The cuff 708 can be configured for placement on or around a medial branch of the hypoglossal nerve or other neural tissue. The lead 704 can optionally include a suture anchor at or near the distal end, at or near the proximal (header) end, or in a central portion thereof.

In an example, the implantable system 602 comprises a cranial nerve stimulator with one or more housings and one or more stimulation leads, and is configured to be implanted in an anterior cervical region, at or near one or more cranial nerves. One or more of the sensor(s) 606 in the implantable system 602 can be configured to sense physiologic signals, sometimes referred to herein as feedback signals. Such physiologic signals, or information therein, can indicate a therapeutic or diagnostic effect. The sensor(s) 606 can be provided inside or outside of the stimulator housing(s) or can be provided on the lead 704. Some examples of the sensor(s) 606 include one or more of an accelerometer 712, motion sensor, acoustic transducer, pressure sensor, optical sensor, photoplethysmography sensor, chemical sensor, electrodes (e.g., on the stimulation lead(s) 608) to sense an ECAP signal or other electrical activity of neural or muscular structures, or one or more other sensors to measure a therapeutic or diagnostic effect.

In an example, a physiologic response that indicates a therapeutic or diagnostic effect can be a function of, or indicated by, one or more of motion, sound, head or neck posture, activity level, force, pressure, vascular changes, pleural cavity changes, electrical activity, bioimpedance change or other information. For example, characteristics of the sensed response can be determined from sensor signal characteristics, for example, using sensor signal information from the time domain (such as a signal amplitude, duration, rise or fall time, slope, period, integral, differential, or other timing characteristic) or from the frequency domain (e.g., signal spectral content).

In an example, physiologic feedback can comprise information about a change in a sensor signal. The feedback can be classified or categorized based on therapeutic effect or diagnostic value. In some examples, therapeutic effect or diagnostic value can be sensed by sensors dedicated to these functions or by sensors that also sense other physiologic information. Using the sensor information or feedback, the nerve stimulator system (e.g., the system 600 from the example of FIG. 6, or a portion thereof) can be used to modulate or titrate therapy automatically or can be used to communicate the sensor information to a clinician via remote or clinic-based follow-up. Subsequently, the clinician can update or titrate the therapy via programmable parameter changes.

In an example, the implantable system 602 of the system 600 includes a hypoglossal nerve (HGN) stimulator configured to treat obstructive sleep apnea. The implantable system 602 can be implanted in a cervical region, such as in one or more of a submental and or submandibular triangle of the neck. The implantable system 602 can include or use one or more stimulation leads placed on or near the hypoglossal nerve(s) of a patient, and can be configured to use electrostimulation therapy to control an upper airway patency-related muscle, for example, the tongue. For example, the implantable system 602 can include one or more electrodes configured to deliver a therapy to induce a change a position of, or to otherwise influence movement of, the tongue, such as to relieve an upper airway obstruction. In some examples, following implantation of the implantable system 602, a clinician adjusts or titrates neurostimulation parameters to achieve a desired tongue movement, such as an excursion of the tongue away from the airway.

Tongue movement, such as in response to an electrostimulation, can manifest as motion, force, pressure, electrical activity (such as an electromyogram signature), bioimpedance change or other effect in various regions of the neck. Furthermore, any resulting upper airway change or obstruction relief can manifest as a change in an acoustic, pressure, or bioimpedance change that can be detected in or near the neck.

In an example, the sensor(s) 606 and/or the processor circuit 610 can be configured to sense or determine diagnostic information such as can include information about a number of apnea or hypopnea events, absence, presence or other characteristics of snoring or other vocalizations, and a general condition of the patient (such as can be indicated by posture or activity level, such as relative to a patient-specific or population-specific reference or baseline). Other information from the sensor(s) 606 and/or the processor circuit 610 can be used to measure tongue movement effects, detect a status of the upper airway, or detect a respiration phase (e.g., inspiration, exhalation, or transitions between inspiration and exhalation), or to use the sensed information to develop a desired or target therapy signature or pattern. The implantable system 602 can optionally be configured to modulate or titrate therapy based on the desired signature or pattern. Alternatively or additionally, the implantable system 602 can be configured to store target therapy signature or pattern information and provide such information via remote or in-clinic follow-up so that a clinician can update the therapy. In an example, historical sensor information can be used to create other signatures or patterns, such as can be used with more recent sensor information to help predict future physiologic events like inspiration timing or breathing interruption.

In an example, the implantable system 602 can include an accelerometer or other sensor configured to measure tongue movement and position. The accelerometer or other sensor can be configured to concurrently sense tracheal sounds or vibrations from the patient's upper airway, such as to monitor for a presence of apnea or hypopnea events, or to detect snoring or other acoustic information, or to identify a phase of the patient's respiration cycle. The accelerometer or other sensor can further be configured to detect orientation of a head, neck, or other body part or to detect posture, or to detect a sleep state. Using information about any one or more of these attributes, the HGN stimulator can develop a signature of a desired physiologic response in a variety of conditions to determine a proper therapy or to determine set of stimulation parameters to be applied to achieve a particular therapy result.

In an example, the HGN stimulator can monitor an accelerometer signal concurrently with, or following, delivery of a stimulation signal, to thereby observe tongue motion or other physiologic response information. In an example, a desired tongue motion can be identified, at least in part, by a rapidly rising or changing acceleration signal, as detected by an accelerometer positioned in the head or cervical region. In an example, less tongue movement can correspond to a diminished accelerometer signal amplitude, while more tongue movement can correspond to a relatively greater signal amplitude. A reference signal amplitude can be associated with a target or desired tongue movement. The HGN stimulator can be configured to automatically adjust a stimulation characteristic (e.g., an amplitude, pulse duration, pulse rate, duty cycle, electrode configuration or other stimulation parameter) to achieve or maintain the target or desired tongue movement. In an example, the HGN stimulator can use information from the accelerometer to determine whether the target or desired tongue movement can be achieved, and can communicate such information to a clinician, such as via remote monitoring for clinical intervention and titration of the therapy.

In an example, one or more of the sensor(s) 606 can be provided superior to, anterior to, or antero-superior to an upper airway obstruction to detect changes in airflow related to obstruction or relief of an obstruction. Sensor placement near or beyond an obstruction (e.g., in a direction away from the lungs) stands in contrast to prior methods that may include sensing information from the chest or thoracic trunk, near the lungs, and thus before the upper airway obstruction. The present inventors have recognized that some signals (sound, pressure, electrical, or otherwise) in the head or neck can be less subject to other (e.g., cardiac) signal interference and can be better correlated with airflow than, e.g., lung sounds. The present inventors have further recognized that some acoustic signals, such can originate from or can be measured at or near the trachea or nearby regions in the head or neck, can have higher amplitude and wider frequency spectrum or content than acoustic signals received at or adjacent to the lungs. The acoustic signals can be measured, for example using the accelerometer 712 or other transducer. The processor circuit 610 can receive the acoustic signals and, based on frequency and magnitude information from the signals, determine whether an airway of the patient is obstructed or partially obstructed. That is, the processor circuit 610 can use acoustic information to indicate an openness of the patient's airway. In an example, the processor circuit 610 can be configured to use the acoustic information to determine whether a patient is snoring, which can be an indication of partial airway obstruction.

In an example, a therapy can be modulated or updated based on patient posture information, such as head or neck posture information. Head or neck posture can be more influential on a patient's apnea hypopnea index (AHI) than, e.g., a chest or trunk posture. Further to modulating therapy based on posture or position, posture information can be used to modulate or help interpret information about tongue motion or information about an ECAP. For example, tongue motion information received from a head-supine position can be interpreted or processed differently than tongue motion information received from a head-lateral position. Similarly, different therapy parameters can be selected or indicated for use depending on a detected posture or position. That is, different electrostimulation parameters can be used to influence or achieve a particular tongue motion when the patient is in a head-supine position, while other electrostimulation parameters can be used to influence or achieve the same tongue motion when the patient is in a head-lateral position.

In an example, the HGN stimulator can be configured to provide automatic neuromodulation by adjusting sets of therapy parameters to achieve target tongue movement, target ECAP response characteristics (e.g., amplitude or timing characteristics), or achieve unobstructed tracheal sounds (e.g., during sleep), such as depending on postural information of the head or neck. The therapy can optionally be determined in a titration sleep study, where the sensor information can be equated to a specific apnea/hypopnea relief signature in a particular patient, as a function of head or neck position or posture.

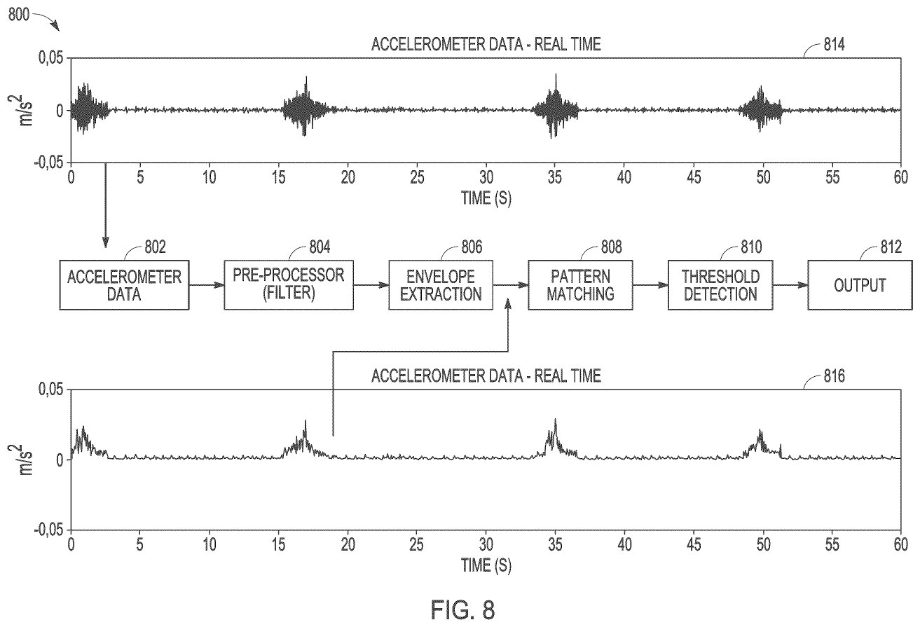

FIG. 8 illustrates generally an example of a physiologic parameter detection algorithm 800 that can include or use an input signal that comprises sensor data from one or more sensor(s) 606. For example, the algorithm can use accelerometer information from the accelerometer 712. In the example of FIG. 8, accelerometer data 802 can include an accelerometer signal 814. The accelerometer signal 814 can include a digital representation of acceleration data corresponding to one or multiple axes. In an example where the accelerometer 712 is implanted in a mandibular, sub-mandibular, cervical, or thoracic region of a patient, the accelerometer data 802 can include information about various physiologic parameters, including respiration, tracheal sounds (breathing), tongue movement, heart rate, patient orientation, and more. The accelerometer data 802 can include data sampled from one or more accelerometers 712 at one or more respective sample rates and data resolutions.

The accelerometer data 802 can be processed using various filters and analyses. For example, the accelerometer data 802 can be used to determine information about patient respiration by pre-processing 804 or filtering the accelerometer data 802. In an example, pre-processing 804 can include band-pass filtering the data with a pass-band of approximately 30 Hz to 60 Hz, or in other pass-bands that include, or are likely to include, information about patient respiration activity.

In an example, pre-processing 804 can include a nth-order (e.g., 4th-order) FIR high-pass Butterworth filter with Fc=30 Hz (or other corner frequency). The filter can help eliminate out-of-band noise, such as may be dominated by 1/f noise. Together with a sampling rate of, e.g., 100 Hz, the filtered result is a signal bandwidth of about 30 Hz to 50 Hz. Removing any DC components in this manner, however, can correspondingly remove information about patient position or orientation. Accordingly, the system can be configured to periodically or intermittently reconfigure its filtering and processing to acquire the orientation information, as further described below.

In an example, the pre-processed accelerometer data 802 can be further processed through envelope extraction 806. In an example, envelope extraction 806 can include rectification and low-pass filtering, such as with a cutoff frequency of, e.g., 1 Hz. FIG. 8 illustrates generally an example of an accelerometer envelope signal 816 for the accelerometer data accelerometer signal 814. In an example, envelope extraction 806 can include or use a Hilbert transform on the results from the pre-processing 804. In another example, envelope extraction 806 can be performed using rectification (e.g., through an absolute value function) and applying a low-pass filter. In an example, the envelope extraction 806 can include or use a smoothing filter, such as can include a 4th order FIR low-pass Butterworth filter with Fc=1 Hz.

In an example, the envelope signal can be further processed using pattern matching 808. The pattern matching 808 can include or use a matched filter with an order of N (e.g., 256). The N filter coefficients can be determined using empirical respiration data collected and averaged to create a template for convolution or matched filtering. In an example, pattern matching 808 can be configured to pattern “match” the filtered accelerometer signal 814 to that of an expected template, and an output of the matched filter can indicate a “match” with a large value output.

In an example, the matched filter comprises a convolution of the template and the sensed signal, where the template includes information about the signal of interest. The output of the matched filter “spikes” when it detects that the template signal exists in the sensed signal.

Additionally or alternatively to pattern matching, a peak-picking technique can be used. In an example, this technique includes tracking a sensed signal over a specified period of time and calculating a mean and standard deviation of the signal values. In an example, the specified period can include several seconds of signal value data, such as 3 seconds. When a current signal value exceeds the historical mean plus standard deviation, then a peak is detected.

The pattern-matched result or peak-detected result can be processed using a threshold detector 810. A threshold (e.g., a patient-specific threshold) can be set and used to identify particular events in the processed or filtered accelerometer signal 814. For example, an output 812 can include a binary indication of whether the filtered accelerometer signal 814 meets or exceeds a specified threshold condition. One or multiple different threshold values can be specified for detecting different peaks or different peak levels in the filtered accelerometer signal 814. In an example, the interface 626 of the external system 620 can be configured to set or update the threshold values.

In an example, the filter parameter or coefficients used for the pattern matching 808 can be updated or adjusted. A patient device can be programmed after implantation (e.g., days or weeks after implantation) and a sleep study can be performed. During this study, the clinician can optimize device settings by monitoring the efficacy of the implant while the patient sleeps. During the study, various polysomnography instruments can be used to monitor the patient to help validate the measurements of the implanted device. For example, pulse, orientation, respiration rate, etc., can all be tracked and can be compared to the values reported by the implant. Differences between physiologic parameter values measured by the implanted device and by other instruments can indicate a need to update or change parameters or coefficients of the template used for the pattern matching 808 in the physiologic parameter detection algorithm 800.

The present inventors have recognized that pattern matching can be computationally expensive and energy intensive. Accordingly, it can be challenging to implement a robust pattern matching algorithm in a low power, implanted device. The present inventors have further recognized that an improved, and less computationally expensive respiration phase detection algorithm can be provided using a threshold detection technique that is based on acceleration signal information from one or more axes of an implanted accelerometer, such as the accelerometer 712.

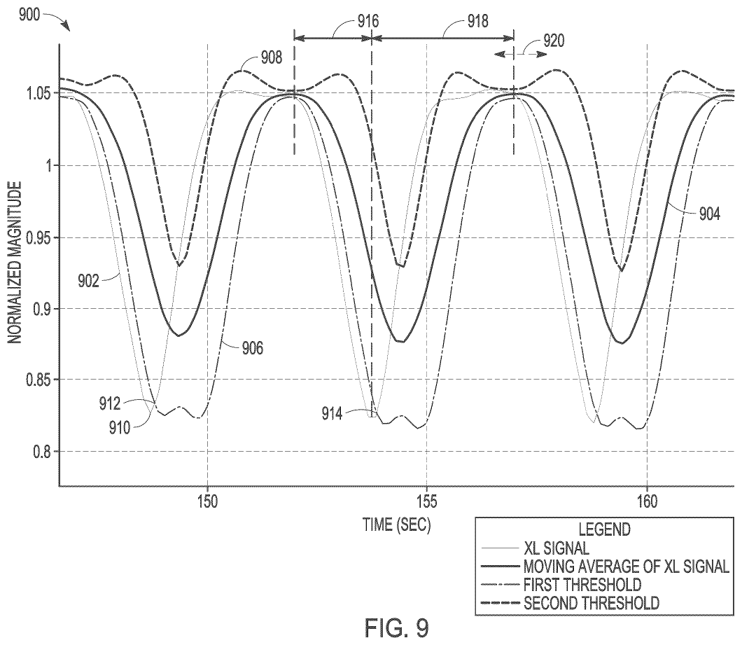

FIG. 9 illustrates generally an example of a first respiration signal chart 900. The first respiration signal chart 900 shows various physiologic signal information over time. In the example of FIG. 9, the first respiration signal chart 900 includes a first acceleration signal 902, a moving average 904 representation of the first acceleration signal 902, a first threshold 906, and a second threshold 908. For purposes of illustration, each of the illustrated traces illustrated in the first respiration signal chart 900 are normalized and time-aligned.

In an example, the first acceleration signal 902 can comprise a signal from the accelerometer 712. In an example, the first acceleration signal 902 comprises a signal from one of multiple axes available from the accelerometer 712, or comprises a composite signal representing information from two or more axes of the accelerometer 712. The system 600 can be configured to select information from an appropriate axis (or combination of axes) based on, for example, a signal to noise characteristic of the first acceleration signal 902, based on a patient orientation or posture, or based on other factors.

As similarly explained above, the accelerometer 712 can comprise a portion of an implantable device that is configured for implantation in a patient body. In an example, the accelerometer 712 can be implanted in a cervical region of the patient. When the accelerometer 712 is implanted in or near the cervical region or submandibular region, the accelerometer 712 can sense motion, physiologic changes or other vibrations that indicate respiration. For example, the accelerometer 712 can be configured to sense acoustic information about an airway of the patient, or the accelerometer 712 can be configured to sense other patient motion that indicates inhalation or exhalation.

The first acceleration signal 902 from the accelerometer 712 can be generally periodic and can correspond to a respiration cycle of the patient. For example, peaks (or valleys, depending on orientation or phase) can indicate transitions between inhalation and exhalation. That is, times of maximum or minimum values of the first acceleration signal 902 can be correlated with an end of inhalation (e.g., when lungs are relatively full of air, just before exhalation) or an end of exhalation (e.g., when the lungs are relatively empty, just before inhalation). Other features of the first acceleration signal 902 (or other physiologic status-indicating signal) can similarly be used to identify timing of respiration phases or transitions between phases.

The present inventors have recognized that peak detection alone may be insufficient to accurately identify respiratory phase transitions between inhalation and exhalation. For example, a single signal that includes information about inhalation and exhalation can have positive and negative extremes and it can be difficult to determine which extreme is associated with which phase. Furthermore, the signal can include other motion or orientation information or signal components that can make it more difficult to reliably extract respiration phase information.

The present inventors have recognized that a solution to the respiratory phase transition detection problem can include establishing or determining one or more time-varying thresholds, and comparing acceleration signal information with the one or more thresholds. A threshold can be based on various statistical measures of an acceleration signal. In an example, a threshold can be based on a moving average of a measured acceleration signal. In an example, a threshold can be based on an interquartile range or standard deviation of a moving average of the measured acceleration signal. The present inventors have recognized that respiratory phase transitions can be more accurately detected by comparing acceleration signal information with a threshold that is based on a statistical characteristic of an acceleration signal, such as a threshold based on a moving standard deviation of the moving average of an acceleration signal.

The example of the first respiration signal chart 900 illustrates detection of respiratory phase transitions using a threshold that is based on statistical characteristics of values of an acceleration signal over time. In the example of FIG. 9, the first respiration signal chart 900 includes a moving average 904 representation of the first acceleration signal 902. The first respiration signal chart 900 further includes a first threshold 906 and a second threshold 908 which can be based on a standard deviation of the moving average 904. For example, the first threshold 906 can be based on the moving average 904 less a standard deviation of the moving average 904, and the second threshold 908 can be based on the moving average 904 plus a standard deviation of the moving average 904.

In an example, a respiration phase transition can be identified based on a relationship between a value of the first acceleration signal 902 and a specified threshold, such as the first threshold 906. That is, a respiration phase transition can be indicated when the first acceleration signal 902 is same-valued or similarly-valued as the first threshold 906. In the example of FIG. 9, a respiration phase transition can coincide with a first signal peak 910 of the first acceleration signal 902. The respiration phase transition can be detected when the first acceleration signal 902 crosses the first threshold 906, such as at a first transition detection time 912 (e.g., around time 149 sec in the example of FIG. 9). In the example, a first signal peak 910 of the first acceleration signal 902 precedes the detected first transition detection time 912 by several milliseconds.

In the example of FIG. 9, the first transition detection time 912 represents a detected time of transition between an inhalation phase and an exhalation phase of a patient's respiratory cycle. That is, at the first transition detection time 912, the patient will have completed (or nearly completed) an inhalation and is beginning to (or will soon begin to) exhale. Subsequent transitions can be similarly identified. For example, a second transition detection time 914 can correspond to a transition from an inhalation phase 916 to an exhalation phase 918 during a respiratory cycle that follows the first transition detection time 912.

In an example, portions of the first acceleration signal 902 can be corrupted or otherwise unusable due to signal noise, patient activity, or other factors. In a particular example, a portion of the first acceleration signal 902 corresponding to an onset of neurostimulation therapy can include information about patient body movement. In an example where the neurostimulation therapy is configured to provide a neurostimulation signal to the hypoglossal nerve 418, such as to induce tongue movement or another muscle response, a portion of the first acceleration signal 902 can include information about tongue movement or other body movement (e.g., motion due to hiccups) and, accordingly, that portion of the first acceleration signal 902 may not be sufficiently representative of respiration. Such corrupted or otherwise unusable respiration information can optionally be discarded or removed from the first acceleration signal 902 before subsequent processing, such as before determining the moving average 904 or before determining one or more of the first threshold 906 and second threshold 908. The information to be discarded can correspond to a blanking duration 920. The information can optionally be replaced, such as by interpolating values of the first acceleration signal 902 before and after the blanking duration 920. The replaced information can optionally be used in subsequent processing, such as to determine the moving average 904 or one or more of the first threshold 906 and second threshold 908. In an example, the blanking duration 920 can begin at an onset of a neurostimulation therapy (e.g., coincident with a beginning of an inhalation phase, or detection of an inhalation phase). The length of the blanking duration 920 can be programmable or dynamically adjustable.

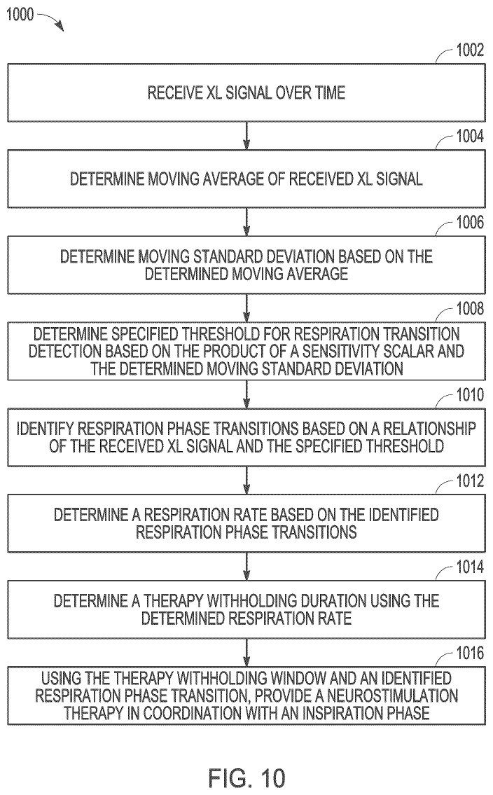

FIG. 10 illustrates an example of a first method 1000 that can include providing a neurostimulation therapy in coordination with an inspiration phase of a respiratory cycle of a patient. Although the example of the first method 1000 shows a particular sequence of operations, the sequence may be altered without departing from the scope of the present disclosure. For example, some of the operations depicted may be performed in parallel or in a different sequence that does not materially affect the function of the first method 1000. In other examples, different components of an example device or system that implements the first method 1000 may perform functions at substantially the same time or in a specific sequence.

At operation 1002, the first method 1000 includes receiving an acceleration signal over time. For example, operation 1002 can include receiving the first acceleration signal 902 from the accelerometer 712 when the accelerometer 712 is implanted in a patient. The accelerometer 712 can be implanted in a location that is susceptible to motion due to respiration of the patient, including in a location where tracheal sounds or other acoustic information can be sensed by the accelerometer 712. In an example, receiving the acceleration signal at operation 1002 includes receiving the acceleration signal at a processor circuit, such as the processor circuit 610 of the implantable system 602 or the processor circuit 624 of the external system 620. In an example, operation 1002 can include filtering (e.g., low-pass filtering) or smoothing the received acceleration signal.

At operation 1004, the first method 1000 includes determining a moving average of the received acceleration signal from operation 1002. In an example, operation 1004 includes determining a Simple Moving Average (SMA). The simple moving average can be calculated by taking an arithmetic mean of a given set of values of the acceleration signal (or a normalized or otherwise pre-processed or filtered version of the acceleration signal) over a specified time duration. For example, a 3 second SMA can be calculated by determining an average value of the acceleration signal over the last 3 seconds. In an example, operation 1004 includes determining an Exponential Moving Average (EMA). The EMA applies more weight to recent signal values in the calculation, such as by using a smoothing factor that gives greater weight to recent data points or signal values and less weight to older data points or signal values. In an example, operation 1004 includes determining a Weighted Moving Average (WMA). The WMA applies more weight to recent data points or signal values by multiplying each data point by a weighting factor that increases, e.g., linearly. The most recent data points or signal values receive the highest weights. In an example, operation 1004 includes determining a Cumulative Moving Average (CMA). The cumulative moving average sums up all the data points or signal values over a specified period and divides the sum by the number of time periods. It gives equal weight to all data points or signal values. In an example, operation 1004 includes determining a Variable Moving Average (VMA). The variable moving average allows the time period for the moving average to vary based on signal volatility or based on specified rules. During periods of high signal volatility, for example, a shorter period may be used.

At operation 1006, the first method 1000 includes determining a moving standard deviation based on the determined moving average from operation 1004. For example, operation 1006 can include computing a simple moving average of the raw acceleration data (e.g., received at operation 1002), then determining deviations from the moving average, squaring and averaging the deviations, and then taking the square root to get the moving standard deviation. In an example, operation 1006 includes a sequence of steps that can be performed on a per-block basis (e.g., every 16 samples, where a group of 16 samples comprises a block). A first step includes calculating a block average (BA_{N}) of all 16 sample values (e.g., of the moving average) in a first block. A second step includes calculating a block difference (BD_{N}) by subtracting a current block average from a previous block average, e.g., BD_{N}=BA_{N}−BA_{N-1}. A third step includes calculating a block difference mean (BDM) across the most recent N blocks (e.g., 16 blocks), where N is a programmable value. For example, Mean=Mean(BD_{N}:BD_{N-15}). A fourth step can include calculating a block difference standard deviation (BDS) across the past 16 (programable) blocks. For example, STDEV=STDEV(BD_{N}:BD_{N-15}). This series of operations can be performed on an ongoing basis to determine the moving standard deviation based on the moving average.

At operation 1008, the first method 1000 includes determining a specified threshold for respiration transition detection. In an example, the specified threshold can be based on the product of a sensitivity scalar (SENS) and the determined moving standard deviation, e.g., from operation 1006. The sensitivity scalar provides adjustable control over the threshold level, enabling the method to robustly detect respiration phase transitions across varying signal conditions and noise characteristics. For example, a high threshold can be computed as: High Threshold=(SENS)*(STDEV) and a low threshold can be computed as Low Threshold=(−SENS)*(STDEV).

In an example, the sensitivity scalar value used in setting the respiration phase transition detection threshold (operation 1008) can be determined using a variety of techniques designed to optimize performance. For example, the sensitivity scalar can be set to an empirically pre-determined constant value that is selected based on testing across a population of patients. In another example, the sensitivity scalar can be tuned on a patient-specific basis by testing a range of values and selecting the one yielding the lowest respiration phase transition detection error rate for that individual. In another example, the sensitivity scalar can be continuously adapted in real-time using a closed-loop control system designed to optimize a performance metric such as respiration rate variability or stimulation timing error.

In an example, the sensitivity scalar can be calculated analytically based on a mathematical model relating the scalar to signal characteristics such as signal noise levels and respiration amplitude. In another example, a machine learning technique such as a neural network can be trained to estimate the optimal sensitivity scalar based on input features like patient characteristics, historic respiration rate, or signal amplitude, among others. In an example, statistical techniques such as maximum likelihood estimation can be used to numerically optimize the sensitivity scalar value based on statistical properties of the respiration acceleration signal.

At operation 1010, the first method 1000 includes identifying one or more respiration phase transitions based on a relationship between the received acceleration signal (e.g., at operation 1002) and the specified threshold (e.g., at operation 1008). When the acceleration signal crosses the specified threshold, a respiration phase transition is detected. Hysteresis or additional signal smoothing functions may be applied to avoid erroneous multiple transition detections from a single respiration event or respiration phase transition. For example, after a transition (e.g., from inhalation to exhalation) is detected, the threshold to detect a subsequent transition can be temporarily adjusted to avoid oscillations. In an example, a minimum refractory period can be enforced between detected transitions to prevent false positives. The minimum refractory period can help reject additional threshold crossings within a specified time window.

In an example, multiple thresholds can be used. For example, respective portions of the acceleration signal can be required to exceed or cross both the first threshold 906 and the second threshold 908 to confirm a particular respiration phase or phase transition.

At operation 1012, the first method 1000 includes determining a respiration rate based on the identified respiration phase transitions. The respiration rate may be averaged or low-pass filtered over a window of several respiration cycles to provide an accurate estimate of respiration rate while smoothing out variability.

In an example, determining the respiration rate can include measuring the time interval between successive inspiration-to-exhalation and exhalation-to-inspiration transitions, and determining the respiration period as the sum of these two interval lengths. In an example, an adaptive filter (e.g., a Kalman filter, or exponential moving average, among others) can be used to estimate a respiration period and its uncertainty. Based on the uncertainty, outlier respiration period measurements may be discarded due to false or missed transitions.

At operation 1014, the first method 1000 includes determining a therapy withholding duration using the determined respiration rate. The withholding duration specifies a time window during which neurostimulation therapy delivery is temporarily withheld following a respiration phase transition. In an example, the withholding window can be expressed as a percentage of the respiration period (e.g., average respiration period over a specified duration). The therapy withholding prevents stimulation during a specified portion of the respiration cycle, such as during exhalation, or at other times when the airway is expected to be unimpeded.

At operation 1016, the first method 1000 includes using the therapy withholding window and an identified respiration phase transition to provide a neurostimulation therapy in coordination with an inspiration phase. For example, following the withholding period after a transition to inspiration, therapy can be delivered during the inspiration phase until the next phase transition, indicating exhalation, is detected.

In an example, the system is configured to use a longer window for averaging respiration rate (e.g., several minutes minutes) to smooth out breath-to-breath variability and detect gradual trends.

In an example, the system is configured to detect and adapt to changes in the patient's breathing rate over time. Multiple adaptive respiration rate estimation techniques operating at different time scales can be used. For example, a short-term estimator can be configured to track respiration rate using a moving average over a window of the most recent detected phase transition intervals. Concurrently, a longer-term estimator can be configured to track a central tendency and variability of respiration rate using a weighted moving average and standard deviation updated continuously over past measurements.

In an example, trends in the amplitude and baseline level of respiratory signals may also be monitored using techniques such as cumulative moving averages and adaptive filters. Detected changes in respiratory signal characteristics can provide information about long-term breathing behavior changes and patient health status changes.

In an example, respiration rate estimates can be analyzed using statistical methods to detect significant departures indicating sustained shifts. In addition, machine learning techniques such as online sequential learning machines can be incorporated to enable real-time adaptive updating of the respiration rate estimators in response to evolving signal characteristics.

By employing parallel respiration rate estimators operating at multiple time scales in conjunction with techniques to detect shifts in respiratory signal patterns, the system can be configured to adapt stimulation timing as patients exhibit both transient and gradual changes in underlying breathing behavior over longer time spans.

In an example, the first method 1000 can include or use inputs from other physiological sensors to detect factors influencing respiratory changes, like patient activity level or sleep stage.

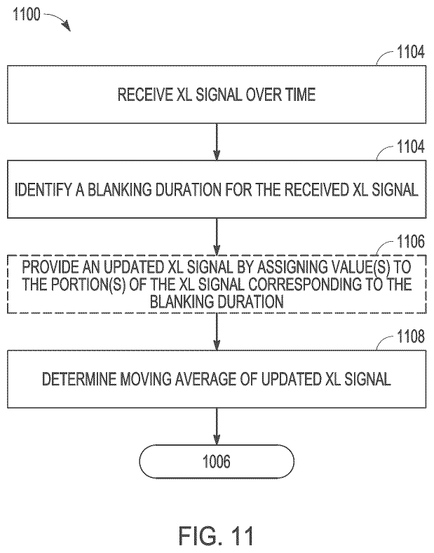

FIG. 11 illustrates an example of a second method 1100 that can include determining a moving average for a physiologic sensor signal. In the example of the second method 1100, the physiologic sensor signal includes an acceleration signal from an accelerometer, such as can be implanted in a cervical region of a patient. Although the example second method 1100 depicts a particular sequence of operations, the sequence may be altered without departing from the scope of the present disclosure. For example, some of the operations depicted may be performed in parallel or in a different sequence that does not materially affect a function of the second method 1100. In other examples, different components of an example device or system that implements the second method 1100 may perform functions at substantially the same time or in a specific sequence.

At operation 1102, the second method 1100 includes receiving an acceleration signal over time. The acceleration signal can be received according to the example of operation 1002 from FIG. 10. Other means or manners of signal acquisition can similarly be used.

At operation 1104, the second method 1100 includes identifying a blanking duration for the received acceleration signal. In an example, operation 1104 can include identifying the blanking duration 920 from the example of FIG. 9. The blanking duration identified at operation 1104 can corresponding to a portion of the information from the acceleration signal (e.g., the signal received at operation 1102) that is to be discarded, disregarded, or replaced, such as prior to evaluation of the acceleration signal.

A length of the blanking duration can be specified, measured, calculated, or otherwise identified. The length can depend at least in part on a respiration rate, on a neurostimulation therapy characteristic, or both. In an example, the blanking duration can be selected to correspond to an actual or expected time interval when patient activity or movement corrupts or otherwise compromises the integrity or fidelity of a physiologic status-indicating component of the acceleration signal. For example, the blanking duration can correspond to a time interval when patient tongue movement compromises the acceleration signal that is received from an accelerometer that is implanted in a cervical region.

In an example, a length of the blanking duration can correspond to a time interval that is specified by a user or clinician. The time interval can be set experimentally, such as during a sleep study, or can have a default value. In an example, the length of the blanking duration can be measured. For example, information from the acceleration signal can be analyzed to identify other patient movement characteristics (e.g., tongue movement) and a duration of the other patient movement can be measured and used as the blanking duration. In an example, the length of the blanking duration can be calculated, for example, based on prior patient or population data. In an example, the length of the blanking duration can be calculated based on an intensity or duration of the neurostimulation therapy that is provided.

In an example, the blanking duration can be determined based on timing information of previous inspiration and stimulation cycles. The blanking duration can be identified by analyzing the frequency content of the acceleration signal and identifying bursts of high frequency activity indicative of patient motion. Signal information like variance, spectral entropy, or autocorrelation can be monitored for changes that can detect the start and end of motion artifacts. In some examples, the blanking duration can be padded or buffered with additional time, such as before and/or after detected or suspected motion, to ensure all artifacts are captured inside the blanking duration.

At operation 1106, the second method 1100 optionally includes providing an updated acceleration signal by assigning one or more values to replace the acceleration signal values during the blanking period. In an example, the assigned values for the acceleration signal can optionally be determined by interpolating values of the received acceleration signal before and after the blanking duration. The replaced information, or assigned values, can be used in subsequent processing.

Functions other than interpolation can be used. For example, a prediction or forecasting model, a regression model, a pattern-matching function or model, or other probabilistic imputation method can be used to generate values that can replace all or a portion of the sensor values that correspond to the blanking duration. The resulting updated acceleration signal can thus include at least a portion of data from the original acceleration signal (e.g., received at operation 1102) and at least a portion of data that corresponds to assigned values. In some examples, the updated acceleration signal includes data from the original acceleration signal without any assigned values or other replacement data corresponding to the blanking duration.

At operation 1108, the example of the second method 1100 includes determining a moving average of the updated acceleration signal, such as the updated acceleration signal provided at operation 1106. The second method 1100 can continue from operation 1108 by advancing to operation 1006 of the first method 1000. For example, continuing the second method 1100 can include using the determined moving average of the updated acceleration signal in subsequent processing, such as to determine one or more thresholds for use in respiration cycle detection.

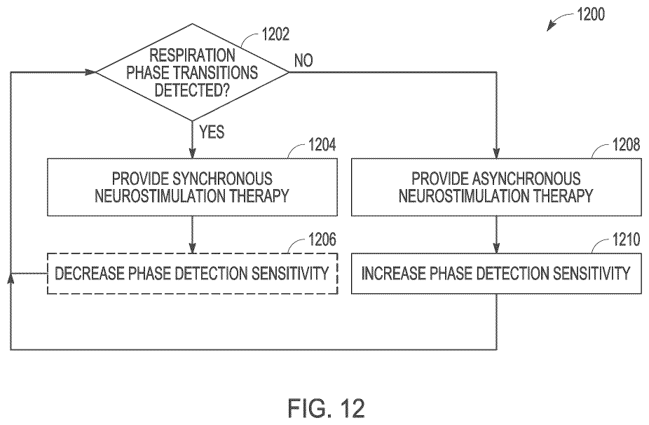

FIG. 12 illustrates generally an example of a third method 1200 that can include selectively providing synchronous neurostimulation therapy or asynchronous neurostimulation therapy. Although the example third method 1200 depicts a particular sequence of operations, the sequence may be altered without departing from the scope of the present disclosure. For example, some of the operations depicted may be performed in parallel or in a different sequence that does not materially affect the function of the third method 1200. In other examples, different components of an example device or system that implements the third method 1200 may perform functions at substantially the same time or in a specific sequence.

At decision block 1202, the third method 1200 includes determining whether two or more respiration phase transitions were detected. In an example, determining whether respiration phase transitions were detected includes or uses at least a portion of the first method 1000 or the second method 1100. For example, respiration phase transitions can be determined using information about a relationship between an acceleration signal and a specified threshold. In an example, decision block 1202 includes determining whether a particular number of respiration phase transitions were detected within a specified time window. The specified time window can depend upon, for example, a measured or expected respiration rate.

If, at decision block 1202, the respiration phase detections were detected, then the third method 1200 can proceed to operation 1204. At operation 1204, the third method 1200 can include providing a synchronous neurostimulation therapy. Providing the synchronous neurostimulation therapy can include delivering neurostimulation pulses in coordination with, or contemporaneously with, some or all of an inspiration phase of a patient's respiratory cycle. That is, the synchronous neurostimulation therapy can begin at or in coordination with a beginning of an inhalation phase, and the therapy can end at or in coordination with an end of the inhalation phase for the same cycle.

In an example, the third method 1200 can optionally include, at operation 1206, decreasing a respiration phase detection sensitivity. Decreasing the sensitivity can help ensure that the synchronous neurostimulation therapy is provided in coordination with at least a portion of the inspiration phase of the patient's respiratory cycle while minimizing power consumption. In an example, decreasing the sensitivity can include changing a threshold value against which the acceleration signal is compared to identify respiration phase transitions. When the sensitivity is reduced, less time is allotted for the inspiration phase. In an example, decreasing the sensitivity can include changing the blanking duration. Accordingly, therapy can be delivered over a lesser period of time, which can in turn improve device battery life and longevity, and reduce overstimulation of the target nerve.

Following a change in the phase detection sensitivity, the third method 1200 can return to decision block 1202 and evaluate whether respiration phase transitions are still detected.

If, at decision block 1202, respiration phase detections are not detected, then the third method 1200 can proceed to operation 1208. At operation 1208, the third method 1200 can include providing an asynchronous neurostimulation therapy. Providing the asynchronous neurostimulation therapy can include delivering neurostimulation pulses intermittently, for example, without regard for the patient's respiratory cycle or phase. Other asynchronous techniques can be used, such as regular but non-phase locked pulse patterns, randomized pulse patterns or pulse delivery timings, or others. The present inventors have recognized that providing an asynchronous therapy can have a therapeutic effect even when the therapy is not delivered in coordination with an inspiration phase of the patient's respiration cycle.

In an example, the third method 1200 can include, at operation 1210, increasing a respiration phase detection sensitivity. Increasing the sensitivity can help increase the likelihood that the phase detection algorithm will successfully identify respiration phase transitions. When respiration phase transitions are successfully identified, then therapy can be provided in coordination with an inspiration phase. In an example, increasing the sensitivity can include changing a threshold value against which the acceleration signal is compared to identify respiration phase transitions. When the sensitivity is increased, more time is allotted for detecting one or both of an inspiration phase and an exhalation phase. In an example, increasing the sensitivity can include changing the blanking duration.

Following a change in the phase detection sensitivity at operation 1210, the third method 1200 can return to decision block 1202 and evaluate whether respiration phase transitions are detected.

Returning to operation 1204, respiration phase transitions may occasionally be undetected due to signal noise or other aberrations. If phase transition sensing lock is lost, then neurostimulation therapy can be delivered at the same cadence as was used before the lost or missing transitions. In other words, recent history of phase transition timings can be used to “fill in” missing detection events and substantially synchronous therapy delivery can continue. If, however, a specified duration elapses without detecting one or more phase transitions, then the third method 1200 can initiate asynchronous therapy delivery at operation 1208.

The adaptive transitioning between synchronous and asynchronous stimulation modes provided by the third method 1200 offers potential benefits and advantages. For example, the third method 1200 provides enhanced therapeutic efficacy by delivering respiration-synchronized neurostimulation when feasible, while still providing asynchronous neurostimulation therapy when respiratory phase locking is not available. The third method 1200 accommodates fluctuations in the sensing availability or efficacy for the patient's respiration by adapting the stimulation mode to changing conditions. In other words, using the third method 1200, therapy delivery can continue using the asynchronous stimulation option if disruptions occur in respiration sensing.

Furthermore, the third method 1200 can be used to help extend the implantable device battery life and longevity using therapy that is timed for delivery only when indicated, or using a reduced duty cycle of asynchronous stimulation. Furthermore, the third method 1200 avoids over-stimulation and optimizes stimulation timing by dynamically adjusting phase detection sensitivity settings, and learning optimal sensitivity thresholds for synchronous stimulation on an individualized patient basis over time.

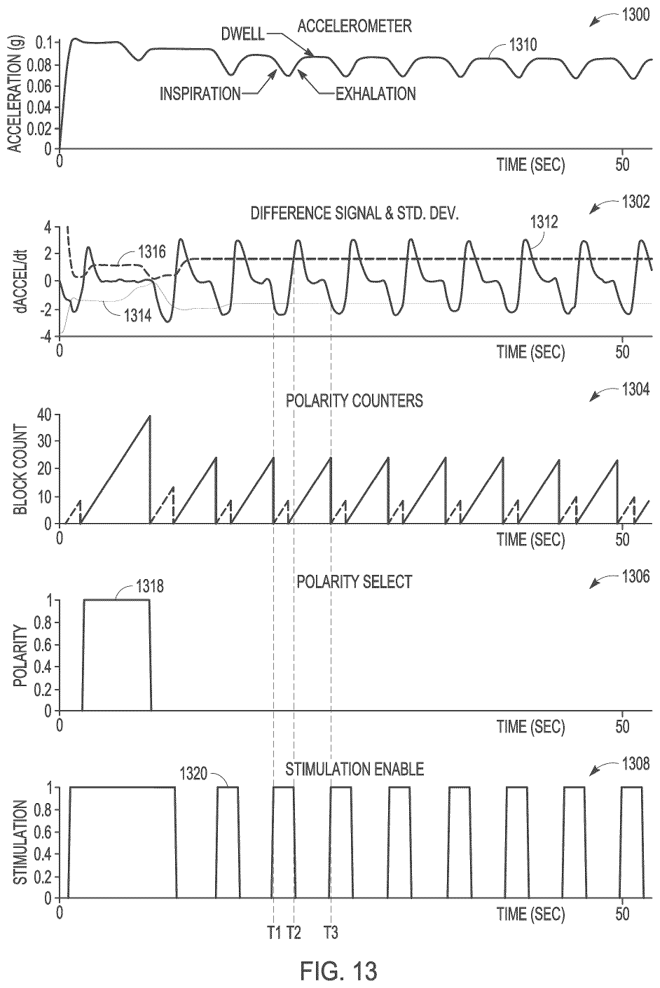

FIG. 13 illustrates generally an example showing a technique for acceleration signal processing for stimulation coordinated with respiration. FIG. 13 includes an acceleration chart 1300, a modified acceleration chart 1302, a polarity counter chart 1304, a polarity selection chart 1306, and a stimulation chart 1308.

In the example of FIG. 13, the acceleration chart 1300 includes a second acceleration signal 1310. The second acceleration signal 1310 can comprise a signal from the accelerometer 712. In an example, the second acceleration signal 1310 comprises a signal from one of multiple axes available from the accelerometer 712, or comprises a composite signal representing information from two or more axes of the accelerometer 712. The system 600 can be configured to select information from an appropriate axis (or combination of axes) based on, for example, a signal to noise characteristic of the acceleration chart 1300, based on a patient orientation or posture, or based on other factors.

The acceleration chart 1300 from the accelerometer 712 can be generally periodic and can correspond to a respiration cycle of the patient. For example, peaks (or valleys, depending on orientation or phase) can indicate transitions between inhalation and exhalation. That is, times of maximum or minimum values of the acceleration chart 1300 can be correlated with an end of inhalation (e.g., when lungs are relatively full of air, just before exhalation) or an end of exhalation (e.g., when the lungs are relatively empty, just before inhalation). As shown in the annotations of the acceleration chart 1300, inspiration or inhalation can coincide with negative slope portions of the second acceleration signal 1310 and exhalation can coincide with positive slope portions of the second acceleration signal 1310. A dwell duration, or pause, between conclusion of expiration and a subsequent inhalation event, can coincide with substantially flat portions of the second acceleration signal 1310. Other features of the second acceleration signal 1310 (or other physiologic status-indicating signal) can similarly be used to identify timing of respiration phases or transitions between phases.

The present inventors have recognized that respiratory phase transition detection can be performed using one or more static or time-varying thresholds, and comparing acceleration signal information, or information that is a function of the acceleration signal information, with the one or more thresholds. The present inventors have further recognized that a jerk signal 1312, or time derivative of the second acceleration signal 1310, can be useful for such a comparison. An example of the jerk signal 1312, based on the second acceleration signal 1310, is illustrated in the modified acceleration chart 1302. A characteristic of the jerk signal 1312 is that its mean value is zero. A further characteristic of the jerk signal 1312 is that each respiratory phase represented by the positive slope and negative slope portions of the second acceleration signal 1310 are instead represented as peaks that can be readily detected using respective threshold comparisons.

A threshold can be based on a statistical measure (e.g., a standard deviation) of a specified portion of the jerk signal 1312. For example, a threshold can optionally be based on a moving standard deviation of the jerk signal 1312. In an example, a standard deviation can be based on the prior N blocks of data that comprise the second acceleration signal 1310 or the jerk signal 1312, where the N blocks represent a sufficient amount of signal information to establish a threshold or boundary condition. In some examples, N blocks represents several seconds (e.g., corresponding to at least one respiration cycle) of sampled data from the accelerometer. The modified acceleration chart 1302 includes a standard deviation-based lower threshold 1314 and a standard deviation-based upper threshold 1316. In the illustrated example, the lower and upper thresholds are based on +/−1 standard deviation from the prior N blocks of data that comprise the jerk signal 1312. The “standard deviation” approach to identifying threshold conditions is discussed herein in particular, illustrative examples. Statistical analysis techniques other than standard deviation-based techniques can similarly be used to identify the various thresholds.

In an example, determining the standard deviation-based thresholds includes a sequence of steps that can be performed on a per-block basis (e.g., every 16 samples, where a group of 16 samples comprises a block). A first step includes calculating a block average (BA_{N}) of all 16 sample values (e.g., jerk signal 1312 values) in a first block. A second step includes calculating a block difference (BD_{N}) by subtracting a current block average from a previous block average, e.g., BD_{N}=BA_{N}−BA_{N-1}. A third step includes calculating a block difference mean (BDM) across the most recent N blocks (e.g., 16 blocks), where N is a programmable value. For example, Mean=Mean(BD_{N}:BD_{N-15}). A fourth step can include calculating a block difference standard deviation (BDS) across the past 16 (programable) blocks. For example, STDEV=STDEV(BD_{N}:BD_{N-15}). This series of operations can be performed on an ongoing basis to determine the moving standard deviation.

Specific thresholds for respiration transition detection can be identified based on the determined standard deviation information. In an example, a threshold can be based on the product of a sensitivity scalar (SENS) and the determined moving standard deviation described above. The sensitivity scalar provides adjustable control over the threshold level, enabling the method to robustly detect respiration phase transitions across varying signal conditions and noise characteristics. For example, the standard deviation-based upper threshold 1316 can be computed as: Upper Threshold=(SENS)*(STDEV) and the standard deviation-based lower threshold 1314 can be computed as Lower Threshold=(−SENS)*(STDEV). These thresholds can be compared to the current block mean (BD_{N}) to determine events for counters, as further described below in the discussion of the polarity counter chart 1304.

In the example of FIG. 13, excursions of the jerk signal 1312 that exceed (e.g., in the negative direction) the standard deviation-based lower threshold 1314 can be understood to coincide with detection of an onset of inspiration, and excursions of the jerk signal 1312 that exceed (e.g., in the positive direction) the standard deviation-based upper threshold 1316 can be understood to coincide with detection of an onset of exhalation. That is, the first time labeled T1 can coincide with an inspiration phase, and the second time labeled T2 can coincide with detection of an onset of, or transition to, exhalation. In an example, the detection points at T1 and T2 can lag the underlying physiologic or physical transition in the respiratory cycle. That is, an actual onset of inspiration can begin a short time before detection of the inspiration phase at T1, such as when the jerk signal 1312 has zero value. Using the threshold comparison approach allows for improved phase detection accuracy at the expense of latency of respiration phase transition identification. A duration from T2 to a third time T3 can represent a portion of the exhalation phase and/or a dwell or pause before onset of the following respiration cycle. In an example, the exhalation phase duration from T2 to T3 includes expiration (i.e., the time when air is leaving the lungs) and the dwell or pause time.

In some examples, such as at the beginning of respiratory phase sensing or following a loss of phase synchronization, it can be unknown whether the positive-going excursions represent inhalation or exhalation, and similarly it can be unknown whether the negative-going excursions represent inhalation or exhalation. The present inventors have recognized, however, that an inspiration phase is generally a smaller duration than an exhalation phase (i.e., an exhalation phase that includes expiration and a pause or dwell time before the next inspiration) and, by measuring the times or durations between threshold crossings, the system can determine which excursions coincide with the respective different phases. That is, by identifying the durations of the different respiratory phases in the time domain, it can be determined which portions of the jerk signal 1312 (and/or the 1310) coincide with inspiration and exhalation.

In an example, a digital counter or other timer can be used to measure the durations. The example of FIG. 13 includes a polarity counter chart 1304 that illustrates the timing of inspiration and exhalation (e.g., expiration and dwell) phases. For example, the polarity counter chart 1304 shows that approximately 10 blocks elapse in the first phase between T1 and T2. The polarity counter chart 1304 shows that approximately 22 blocks elapse in the second phase between T2 and T3. In this example, the second phase is longer than the first phase by a factor of about 2.2:1. Since an inspiration phase is generally about half of the duration of an exhalation phase, the first phase can be identified as the inspiration phase, and the second phase can be identified as the exhalation phase.

The example of FIG. 13 includes a polarity selection chart 1306. The polarity selection chart 1306 includes a logical or binary polarity signal 1318 that indicates whether negative-going excursions are determined to coincide with inspiration or exhalation. That is, a low-valued polarity signal (e.g., “0”) can indicate that the negative-going excursion of the jerk signal 1312 below the standard deviation-based lower threshold 1314 at T1 represents inspiration and, accordingly, that the positive-going excursion of the jerk signal 1312 above the standard deviation-based upper threshold 1316 at T2 represents an onset of exhalation. In contrast, a high-valued polarity signal (e.g., “1”) can indicate that the negative-going excursion of the jerk signal 1312 below the standard deviation-based lower threshold 1314 represents an onset of exhalation. The polarity signal can thus be a function of various logic circuitry that receives, at an input, the count or timing information represented in the polarity counter chart 1304, and that provides, at an output, a representation of which respiratory phase coincides with each threshold crossing of the jerk signal 1312.

The example of FIG. 13 includes a stimulation chart 1308 with a logical or binary stimulation enable signal 1320. The stimulation enable signal 1320 represents when a neurostimulation therapy is provided to a patient. For example, the neurostimulation therapy can be provided in coordination with a patient's respiratory cycle, such as during an inhalation phase. Therapy can be turned off or withheld during the patient's exhalation phase. In the example of the stimulation chart 1308, the stimulation enable signal 1320 indicates that therapy is enabled when the signal is high, and indicates therapy is off or disabled when the signal is low. For example, therapy can be enabled between times T1 and T2, and therapy can be disabled between times T2 and T3.

In an example, portions of the second acceleration signal 1310 can be corrupted or otherwise unusable due to signal noise, patient activity, or other factors. In a particular example, a portion of the second acceleration signal 1310 corresponding to an onset of neurostimulation therapy can include information about patient body movement. In an example where the neurostimulation therapy is configured to provide a neurostimulation signal to the hypoglossal nerve 418, such as to induce tongue movement or another muscle response, a portion of the second acceleration signal 1310 can include information about tongue movement or other body movement (e.g., motion due to hiccups) and, accordingly, that portion of the second acceleration signal 1310 may not sufficiently represent respiration. Such corrupted or otherwise unusable respiration information can optionally be discarded or removed from the second acceleration signal 1310 before subsequent processing, such as before determining the jerk signal 1312 or before determining one or more of the thresholds based on the jerk signal 1312. The information to be discarded can correspond to a blanking duration and can optionally be replaced, such as by interpolating values of the acceleration signal before and after the blanking duration. The replaced information can optionally be used in subsequent processing, such as to determine the jerk signal 1312 or one or more of the thresholds.

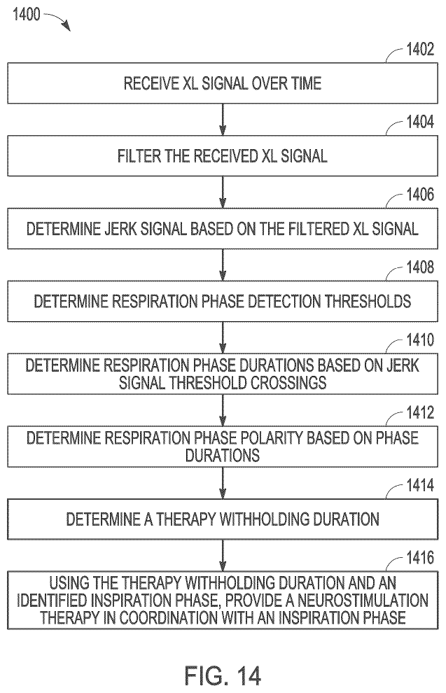

FIG. 14 illustrates an example of a fourth method 1400 that can include providing a neurostimulation therapy in coordination with an inspiration phase of a respiratory cycle of a patient. Although the example of the fourth method 1400 shows a particular sequence of operations, the sequence may be altered without departing from the scope of the present disclosure. For example, some of the operations depicted may be performed in parallel or in a different sequence that does not materially affect the function of the fourth method 1400. In other examples, different components of an example device or system that implements the fourth method 1400 may perform functions at substantially the same time or in a specific sequence.

At operation 1402, the fourth method 1400 includes receiving an acceleration signal over time. For example, operation 1402 can include receiving the second acceleration signal 1310 from the accelerometer 712 when the accelerometer 712 is implanted in a patient. The accelerometer 712 can be implanted in a location that moves due to respiration of the patient, optionally including in a location where tracheal sounds or other acoustic information can be sensed by the accelerometer 712. In an example, receiving the acceleration signal at operation 1402 includes receiving the acceleration signal at a processor circuit, such as the processor circuit 610 of the implantable system 602 or the processor circuit 624 of the external system 620. In an example, operation 1402 can include filtering (e.g., low-pass filtering) or smoothing the received acceleration signal. In an example, the acceleration signal can be digitally sampled, such as at a sample rate of about 100 samples per second, and 16 bits per sample. In an example, the samples can be managed in blocks of samples, e.g., 16 samples. In this example, the block rate is 100/16 Hz.

At operation 1404, the fourth method 1400 includes filtering the acceleration signal received at operation 1402. Filtering the signal can include, for example, using an IIR DFT low-pass filter to remove noise or optimize the signal for subsequent processing.

At operation 1406, the fourth method 1400 includes determining a jerk signal based on the filtered acceleration signal from operation 1404. Accelerometers measure the rate of change of velocity, or acceleration, across one or more axes (x, y, z), producing a signal that represents acceleration as a function of time. Taking the first derivative of the acceleration signal yields the “jerk” (sometimes called “jolt”), a vector quantity that describes how acceleration changes over time. Jerk can be measured in, for example, meters per second cubed (m/s³), such as when acceleration is measured in meters per second squared (m/s²). Mathematically, if (a(t)) denotes the acceleration signal as a function of time, the jerk (j(t)) can be expressed as j(t)=da(t)/dt, representing the rate of change of acceleration with respect to time.

The jerk signal is significant because it provides insights into the dynamics of motion, indicating how smoothly or abruptly acceleration changes. High jerk values suggest rapid changes in acceleration, such as sudden starts or stops, or transitions between inhalation and exhalation. Low values, in contrast, indicate smoother transitions. Jerk signals can be analyzed in time and frequency domains using signal processing techniques like filtering and Fourier analysis to reduce noise and extract relevant features.

In an example, determining the jerk signal at operation 1406 includes calculating a mean of a current sample block, and subtracting it from the mean of the previous block. The previous M block differences can be stored and used for further statistical analysis. In an example, M is an integer, such as 16 or 32, and the stored block difference information corresponds to sample information from the previous 2-6 seconds. Other block sizes can similarly be used.

At operation 1408, the fourth method 1400 includes determining respiration phase detection thresholds. The operation 1408 can include calculating a mean and standard deviation of the most recent R stored samples. In some examples, R is an integer equal to M. The operation 1408 can include determining an upper threshold (e.g., the standard deviation-based upper threshold 1316) based on the mean plus a standard deviation of the R samples, and can include determining a lower threshold (e.g., the standard deviation-based lower threshold 1314) based on the mean less a standard deviation of the R samples.

In an example, the upper and/or lower thresholds can be adjusted to accommodate different levels of sensitivity in respiration phase sensing. That is, the threshold(s) can be based on the product of a sensitivity scalar and the determined standard deviation-based detection thresholds, e.g., from operation 1408. The sensitivity scalar provides adjustable control over a threshold level, enabling the method to detect respiration phase transitions across varying signal conditions and noise characteristics.

In an example, the sensitivity scalar value used in setting the respiration phase transition detection threshold can be determined using a variety of techniques designed to optimize performance. For example, the sensitivity scalar can be set to an empirically pre-determined constant value that is selected based on testing across a population of patients. In another example, the sensitivity scalar can be tuned on a patient-specific basis by testing a range of values and selecting the one yielding the lowest respiration phase transition detection error rate for that individual. In another example, the sensitivity scalar can be continuously adapted in real-time using a closed-loop control system designed to optimize a performance metric such as respiration rate variability or stimulation timing error.

In an example, the sensitivity scalar can be calculated analytically based on a mathematical model relating the scalar to signal characteristics such as signal noise levels and respiration amplitude. In another example, a machine learning technique such as a neural network can be trained to estimate the optimal sensitivity scalar based on input features like patient characteristics, historic respiration rate, or signal amplitude, among others. In an example, statistical techniques such as maximum likelihood estimation can be used to numerically optimize the sensitivity scalar value based on statistical properties of the respiration acceleration signal.

In an example, operation 1408 includes determining the phase detection threshold(s) based on a moving standard deviation. For example, operation 1408 can include computing a simple moving average of the jerk signal (e.g., determined at operation 1406) and determining deviations from the moving average, squaring and averaging the deviations, and then taking the square root to provide a moving standard deviation.

At operation 1410, the fourth method 1400 includes determining respiration phase durations based on a relationship between the jerk signal and the phase detection thresholds provided at operation 1408. For example, an onset of a first respiration phase can be identified at a first crossing of the jerk signal and a first one of the upper and lower thresholds. In the example of FIG. 13, the first crossing is at time T1 when the jerk signal 1312 meets the standard deviation-based lower threshold 1314. A conclusion of the first respiration phase coincides with an onset of a second respiration phase, and can be identified at a subsequent crossing of the jerk signal and the other one of the upper and lower thresholds. In the example of FIG. 13, the subsequent crossing is at time T2 when the jerk signal 1312 meets the standard deviation-based upper threshold 1316. A conclusion of the second respiration phase coincides with an onset of a third respiration phase and can be identified at a further subsequent crossing of the jerk signal and its next threshold crossing. In the example of FIG. 13, the further subsequent crossing is at time T3 when the jerk signal 1312 again meets the standard deviation-based lower threshold 1314. The operation 1410 can include measuring a duration of the first respiration phase (e.g., the duration between T1 and T2) and can include measuring a duration of the second respiration phase (e.g., the duration between T2 and T3).

At operation 1412, the fourth method 1400 includes determining a respiration phase polarity based on the phase duration information from operation 1410. In an example, operation 1412 includes comparing the duration of the first respiration phase (e.g., the duration between T1 and T2) and the duration of the second respiration phase (e.g., the duration between T2 and T3). The phase with the shorter duration can be identified as an inspiration phase and the phase with the longer duration can be identified as an exhalation phase.

At operation 1414, the fourth method 1400 can include determining a therapy withholding duration. The withholding duration specifies a time window during which neurostimulation therapy delivery is temporarily withheld following a respiration phase transition. In an example, the withholding window can be expressed as a percentage of the respiration period (e.g., average respiration period over a specified duration). The therapy withholding prevents stimulation during a specified portion of the respiration cycle, such as during exhalation, or at other times when the airway is expected to be unimpeded.

In an example, operation 1414 includes using a specified withholding duration, such as can be specified by a patient or clinician. For example, the withholding duration can correspond to a measured or observed duration from a patient sleep study. In another example, the withholding duration can be based on a determined respiration rate. Determining the respiration rate can include measuring the time interval between successive inspiration-to-exhalation and exhalation-to-inspiration transitions, and determining the respiration period as the sum of these two interval lengths. In an example, an adaptive filter (e.g., a Kalman filter, or exponential moving average, among others) can be used to estimate a respiration period and its uncertainty. Based on the uncertainty, outlier respiration period measurements may be discarded due to false or missed transitions.

At operation 1416, the fourth method 1400 can include using the therapy withholding duration from operation 1414 and providing a neurostimulation therapy in coordination with an inspiration phase of a patient's respiratory cycle. For example, following a withholding period that begins at an onset of exhalation, therapy can be delivered during the inspiration phase until the next phase transition, indicating exhalation, is detected. In an example, the therapy delivery can include a specified minimum duration of therapy or minimum duty cycle, and can include a minimum off time (or withholding period). By imposing minimum therapy on and off durations, the system can avoid stimulation at a high duty cycle. High duty cycle stimulation can, under some circumstances, have limited therapeutic benefit and unnecessarily consume system power.

In an example, the system is configured to detect and adapt to changes in the patient's breathing rate over time. One or more adaptive respiration rate estimation techniques operating at different time scales can be used. For example, a short-term estimator can be configured to track respiration rate using a moving average over a window of the most recent detected phase transition intervals. Concurrently, a longer-term estimator can be configured to track a central tendency and variability of respiration rate using a weighted moving average and standard deviation updated continuously over past measurements.

In an example, trends in the amplitude and baseline level of respiratory signals can be monitored using techniques such as cumulative moving averages and adaptive filters. Detected changes in respiratory signal characteristics can provide information about long-term breathing behavior changes and patient health status changes.

In an example, respiration rate estimates can be analyzed using statistical methods to detect significant departures indicating sustained shifts. In addition, machine learning techniques such as online sequential learning machines can be incorporated to enable real-time adaptive updating of the respiration rate estimators in response to evolving signal characteristics.

By employing parallel respiration rate estimators operating at multiple time scales in conjunction with techniques to detect shifts in respiratory signal patterns, the system can be configured to adapt stimulation timing as patients exhibit both transient and gradual changes in underlying breathing behavior over longer time spans.

In an example, the fourth method 1400 can include or use inputs from other physiological sensors to detect factors influencing respiratory changes, like patient activity level or sleep stage.

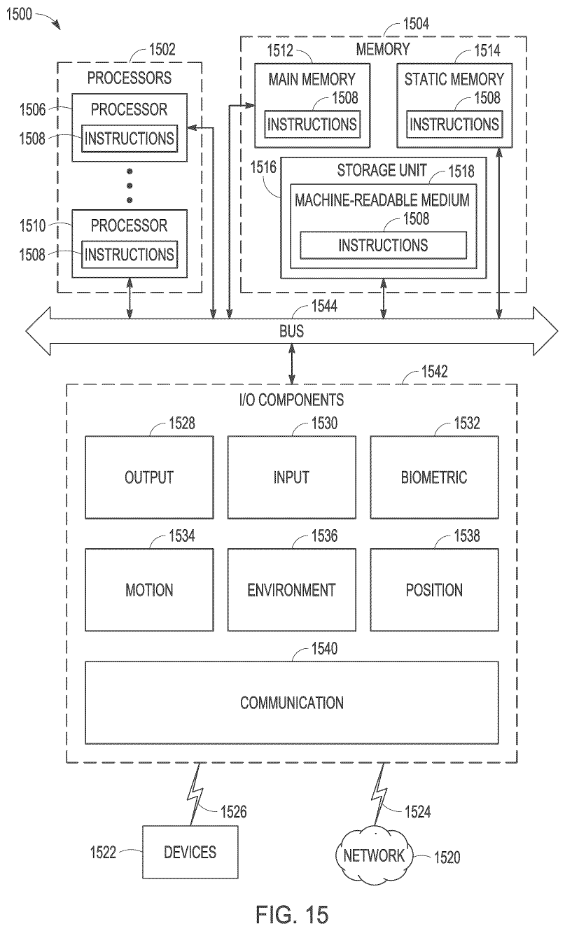

FIG. 15 is a diagrammatic representation of a machine 1500 within which instructions 1508 (e.g., software, a program, an application, an applet, an app, or other executable code) for causing the machine 1500 to perform any one or more of the methodologies discussed herein may be executed. The machine 1500 can optionally comprise the implantable system 602, the external system 620, or components or portions thereof, or components or devices that can be coupled to at least one of the implantable system 602 and the external system 620.

In an example, the instructions 1508 may cause the machine 1500 to execute any one or more of the methods, controls, therapy algorithms, signal processing algorithms, signal generation routines, or other processes described herein. The instructions 1508 transform the general, non-programmed machine 1500 into a particular machine 1500 programmed to carry out the described and illustrated functions in the manner described. The machine 1500 may operate as a standalone device or may be coupled (e.g., networked) to other machines. In a networked deployment, the machine 1500 may operate in the capacity of a server machine or a client machine in a server-client network environment, or as a peer machine in a peer-to-peer (or distributed) network environment. The machine 1500 can comprise, but is not limited to, various systems or devices that can communicate with the implantable system 602 or the external system 620, such as can include a server computer, a client computer, a personal computer (PC), a tablet computer, a laptop computer, a netbook, a set-top box (STB), a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 1508, sequentially or otherwise, that specify actions to be taken by the machine 1500. Further, while only a single machine 1500 is illustrated, the term “machine” shall also be taken to include a collection of machines that individually or jointly execute the instructions 1508 to perform any one or more of the methodologies discussed herein.

The machine 1500 may include processors 1502, memory 1504, and I/O components 1542, which may be configured to communicate with each other via a bus 1544. In an example embodiment, the processors 1502 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an ASIC, a Radio-Frequency Integrated Circuit (RFIC), another processor, or any suitable combination thereof) may include, for example, a processor 1506 and a processor 1510 that execute the instructions 1508. The term “processor” is intended to optionally include multi-core processors that may comprise two or more independent processors (sometimes referred to as “cores”) that may execute instructions contemporaneously. Although FIG. 15 shows multiple processors 1502, the machine 1500 may include a single processor with a single core, a single processor with multiple cores (e.g., a multi-core processor), multiple processors with a single core, multiple processors with multiples cores, or any combination thereof.

The memory 1504 includes a main memory 1512, a static memory 1514, and a storage unit 1516, both accessible to the processors 1502 via the bus 1544. The main memory 1504, the static memory 1514, and storage unit 1516 store the instructions 1508 embodying any one or more of the methodologies or functions described herein. The instructions 1508 may also reside, completely or partially, within the main memory 1512, within the static memory 1514, within a machine-readable medium 1518 within the storage unit 1516, within at least one of the processors 1502 (e.g., within the processor's cache memory), or any suitable combination thereof, during execution thereof by the machine 1500.

The I/O components 1542 may include a variety of components to receive input, provide output, produce output, transmit information, exchange information, capture measurements, and so on. The specific I/O components 1542 that are included in a particular machine will depend on the type of machine. For example, portable machines such as device programmers or mobile phones may include a touch input device or other such input mechanisms, while a headless server machine will likely not include such a touch input device. It will be appreciated that the I/O components 1542 may include other components that are not shown in FIG. 15. In various example embodiments, the I/O components 1542 may include output components 1528 and input components 1530. The output components 1528 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 1530 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), physiologic sensor components, and the like.

In further example embodiments, the I/O components 1542 may include biometric components 1532, motion components 1534, environmental components 1536, or position components 1538, among others. For example, the biometric components 1532 can include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The motion components 1534 can include an acceleration sensor (e.g., an accelerometer configured to measure acceleration about one or more axes), gravitation sensor components, rotation sensor components (e.g., a gyroscope), or similar. The environmental components 1536 can include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 1538 can include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 1542 further include communication components 1540 operable to couple the machine 1500 to a network 1520 or other devices 1522 via a coupling 1524 and a coupling 1526, respectively. For example, the communication components 1540 may include a network interface component or another suitable device to interface with the network 1520. In further examples, the communication components 1540 may include wired communication components, wireless communication components, cellular communication components, Near Field Communication (NFC) components, Bluetooth components, or Wi-Fi components, among others. The devices 1522 may be another machine or any of a wide variety of peripheral devices such as can include other implantable or external devices.

The various memories (e.g., memory 1504, main memory 1512, static memory 1514, and/or memory of the processors 1502) and/or storage unit 1516 can store one or more sets of instructions and data structures (e.g., software) embodying or used by any one or more of the methodologies or functions described herein. These instructions (e.g., the instructions 1508), when executed by processors 1502, cause various operations to implement the disclosed embodiments, including various neuromodulation or neurostimulation therapies or functions supportive thereof.

To better illustrate the systems and methods described herein, such as can be used to optimize an electrostimulation therapy to treat a sleep disorder or breathing disorder for a patient, a non-limiting set of Example embodiments are set forth below as numerically identified Examples.

Example 1 is a method for controlling delivery of a neurostimulation therapy, the method comprising: identifying an onset of an exhalation phase of a respiratory cycle of a patient; initiating a timer at the onset of the exhalation phase, wherein the timer is configured to identify expiration of a therapy withholding duration; and in response to the expiration of the therapy withholding duration, providing the neurostimulation therapy in coordination with an onset of an inspiration phase of the respiratory cycle of the patient.

In Example 2, the subject matter of Example 1 optionally includes the therapy withholding duration is based on a sensed respiratory rate of the patient.

In Example 3, the subject matter of Example 2 optionally includes sensing the respiratory rate of the patient using an acceleration signal from an accelerometer when the accelerometer is implanted in a submandibular region of the patient.

In Example 4, the subject matter of Example 3 optionally includes sensing the respiratory rate of the patient, including: determining a moving average of the acceleration signal over a first duration; determining a moving standard deviation based on the moving average; determining a first specified threshold based on the moving standard deviation; identifying respiration phase transitions based on a relationship between the first specified threshold and the acceleration signal; and determining the respiratory rate based on the identified respiration phase transitions.

In Example 5, the subject matter of Example 4 optionally includes identifying the respiration phase transitions including identifying respective times when a value of the acceleration signal meets or exceeds the first specified threshold.

In Example 6, the subject matter of any one or more of Examples 1-5 optionally includes determining the therapy withholding duration based on a measured duration of an exhalation phase and a blanking duration.

In Example 7, the subject matter of Example 6 optionally includes sensing the respiratory rate of the patient using an acceleration signal from an accelerometer when the accelerometer is implanted in a submandibular region of the patient; and determining the blanking duration based on interpolated values of the acceleration signal.

Example 8 is a method for controlling delivery of a neurostimulation therapy, the method comprising: receiving an acceleration signal from an accelerometer, wherein the accelerometer is configured for implantation in a submandibular region or cervical region of a patient; determining a jerk signal based on the acceleration signal; determining a standard deviation based on the jerk signal; determining first and second specified thresholds based on the standard deviation; identifying a first respiration phase based on a relationship between the jerk signal and the first and second specified thresholds, and identifying a subsequent second respiration phase based on a relationship between the jerk signal and the second and first specified thresholds; identifying, as a reference inspiration phase, one of the first and second respiration phases having a shorter duration; and providing the neurostimulation therapy in coordination with an onset of a subsequent inspiration phase of a respiratory cycle of the patient, wherein the onset of the subsequent inspiration phase follows the reference inspiration phase and a therapy withholding duration.

In Example 9, the subject matter of Example 8 optionally includes determining a respiration rate based on timing characteristics of the first and subsequent second respiration phases; and determining the therapy withholding duration based on the determined respiration rate.

In Example 10, the subject matter of Example 9 optionally includes monitoring the respiration rate over time; and in response to a detected change in the respiration rate, changing the therapy withholding duration.

In Example 11, the subject matter of any one or more of Examples 8-10 optionally includes the first specified threshold is based on a product of the standard deviation and a specified sensitivity scalar.

In Example 12, the subject matter of any one or more of Examples 8-11 optionally includes identifying the first respiration phase including identifying a first time when a value of the jerk signal meets the first specified threshold and identifying a second time when a subsequent value of the jerk signal meets the second specified threshold.

Example 13 is a method for controlling delivery of a neurostimulation therapy, the method comprising: receiving an acceleration signal from an implanted accelerometer; in response to identifying a first series of respiration phase transition events based on information from the acceleration signal, providing the neurostimulation therapy synchronously with an inspiration phase of a patient's respiratory cycle; and in absence of identifying the first series of respiration phase transition events in the acceleration signal, providing the neurostimulation therapy asynchronously with the patient's respiratory cycle.

In Example 14, the subject matter of Example 13 optionally includes providing the neurostimulation therapy synchronously with the inspiration phase of the patient's respiratory cycle including providing the neurostimulation therapy during the inspiration phase without providing the neurostimulation therapy during one or more other phases of the patient's respiratory cycle.

In Example 15, the subject matter of any one or more of Examples 13-14 optionally includes providing the neurostimulation therapy asynchronously with the patient's respiratory cycle including providing the neurostimulation therapy intermittently throughout the patient's respiratory cycle.

In Example 16, the subject matter of any one or more of Examples 13-15 optionally includes identifying the first series of respiration phase transition events including: determining a jerk signal based on the acceleration signal; determining upper and lower thresholds based on a standard deviation of the jerk signal; identifying respective phase transition events over time based on a relationship between values of the jerk signal and the upper and lower thresholds.

In Example 17, the subject matter of Example 16 optionally includes adjusting the upper and/or lower thresholds to change a sensitivity of the identification of the phase transition events.

In Example 18, the subject matter of Example 17 optionally includes decreasing the sensitivity in response to providing the neurostimulation therapy synchronously with the patient's respiratory cycle.

In Example 19, the subject matter of any one or more of Examples 17-18 optionally includes increasing the sensitivity in response to providing the neurostimulation therapy asynchronously with the patient's respiratory cycle.

In Example 20, the subject matter of any one or more of Examples 13-19 optionally includes identifying the first series of respiration phase transition events including: determining a moving average of the acceleration signal over a first duration; determining a moving standard deviation based on the moving average; determining a first specified threshold based on the moving standard deviation; and identifying respective respiration phase transition events over time based on a relationship between the first specified threshold and the acceleration signal.

In Example 21, the subject matter of Example 20 optionally includes adjusting the first specified threshold to thereby change a sensitivity of the identification of the respective respiration phase transition events.

In Example 22, the subject matter of Example 21 optionally includes decreasing the sensitivity in response to providing the neurostimulation therapy synchronously with the patient's respiratory cycle.

In Example 23, the subject matter of any one or more of Examples 21-22 optionally includes increasing the sensitivity in response to providing the neurostimulation therapy asynchronously with the patient's respiratory cycle.

Example 24 is a system comprising: a first housing configured for implantation in a submandibular region or cervical region of a patient; a first electrode lead coupled to the first housing and configured to be disposed in a submandibular region, wherein at least one electrode on the first electrode lead is configured to be disposed at or near a first branch of a hypoglossal nerve of the patient to provide a first neurostimulation therapy that is configured to treat a sleep disorder or breathing disorder of the patient; an accelerometer configured to provide an acceleration signal that includes information about a respiration cycle of the patient; and a processor circuit configured to: receive the acceleration signal; determine a jerk signal based on the acceleration signal; determine a standard deviation based on the jerk signal; determine first and second specified thresholds based on the standard deviation; identify respiration phase transitions based on a relationship between the jerk signal and the first and second specified thresholds; and provide a control signal to a signal generator circuit to provide the first neurostimulation therapy in coordination with an onset of an inspiration phase of a respiratory cycle of the patient, wherein the onset of the inspiration phase is determined based on the identified respiration phase transitions.

In Example 25, the subject matter of Example 24 optionally includes the processor circuit is configured to: determine a respiration rate based on the identified respiration phase transitions; determine a therapy withholding duration based on the respiration rate; and wherein the onset of the inspiration phase follows a first respiration phase transition, of the identified respiration phase transitions, by the therapy withholding duration.

In Example 26, the subject matter of any one or more of Examples 24-25 optionally includes the processor circuit is configured to: measure respective durations between the identified respiration phase transitions; and identify an inspiration phase of the respiratory cycle as a shorter duration phase adjacent to a longer duration phase.

Example 27 is a method for controlling delivery of a neurostimulation therapy, the method comprising: receiving an acceleration signal from an accelerometer, wherein the accelerometer is configured for implantation in a submandibular region or cervical region of a patient; determining a moving average of the acceleration signal over a first duration; determining a moving standard deviation based on the moving average; determining a first specified threshold based on the moving standard deviation; identifying respiration phase transitions based on a relationship between the first specified threshold and the acceleration signal; determining a respiration rate based on the identified respiration phase transitions; determining a therapy withholding duration based on the respiration rate; providing the neurostimulation therapy in coordination with an onset of an inspiration phase of a respiratory cycle of the patient, wherein the onset of the inspiration phase follows a first respiration phase transition, of the identified respiration phase transitions, by the therapy withholding duration.

In Example 28, the subject matter of Example 27 optionally includes the first specified threshold is based on a product of the moving standard deviation and a specified sensitivity scalar.

In Example 29, the subject matter of any one or more of Examples 27-28 optionally includes identifying the respiration phase transitions including identifying respective times when a value of the acceleration signal meets or exceeds the first specified threshold.

In Example 30, the subject matter of Example 29 optionally includes identifying the respiration phase transitions including identifying respective times corresponding to an onset of exhalation.

In Example 31, the subject matter of any one or more of Examples 27-30 optionally includes monitoring the respiration rate over time; and in response to a detected change in the respiration rate, changing the therapy withholding duration.

In Example 32, the subject matter of any one or more of Examples 27-31 optionally includes identifying the respiration phase transitions including changing the first specified threshold to increase a likelihood that a value of the acceleration signal meets or exceeds the first specified threshold.

In Example 33, the subject matter of any one or more of Examples 27-32 optionally includes determining the moving average of the acceleration signal including identifying one or more blanking durations corresponding to one or more portions of the acceleration signal over the first duration, and determining the moving average without using the one or more portions of the acceleration signal that correspond to the one or more blanking durations.

In Example 34, the subject matter of Example 33 optionally includes determining likely values of the acceleration signal for the one or more blanking durations; and wherein determining the moving average of the acceleration signal includes using the determined likely values.

In Example 35, the subject matter of any one or more of Examples 33-34 optionally includes determining the likely values of the acceleration signal including using an interpolation function based on values of the acceleration signal outside of the blanking durations.

Example 36 is a system comprising: a first housing configured for implantation in a submandibular region or cervical region of a patient; a first electrode lead coupled to the first housing and configured to be disposed in a submandibular region, wherein at least one electrode on the first electrode lead is configured to be disposed at or near a first branch of a hypoglossal nerve of the patient to provide a first neurostimulation therapy that is configured to treat a sleep disorder or breathing disorder of the patient; an accelerometer configured to provide an acceleration signal that includes, information about a respiration cycle of the patient; and a processor circuit configured to: receive the acceleration signal; determine a moving average of the acceleration signal over a first duration; determine a moving standard deviation based on the moving average; determine a first specified threshold based on the moving standard deviation; identify respiration phase transitions based on a relationship between the first specified threshold and the acceleration signal; determine a respiration rate based on the identified respiration phase transitions; determine a therapy withholding duration based on the respiration rate; and provide a control signal to a signal generator circuit to provide the first neurostimulation therapy in coordination with an onset of an inspiration phase of a respiratory cycle of the patient, wherein the onset of the inspiration phase follows a first respiration phase transition, of the identified respiration phase transitions, by the therapy withholding duration.

Example 37 is at least one tangible, non-transitory machine-readable medium including instructions that, when executed by processing circuitry, cause the processing circuitry to perform operations to implement any of Examples 1-36.

Each of these non-limiting examples can stand on its own, or can be combined in various permutations or combinations with one or more of the other examples.

The above description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part, such as using the implantable system 602, the external system 620, the machine 1500, or using the other systems, devices, or components discussed herein. Some examples can include a computer-readable medium or machine-readable medium encoded with instructions operable to configure an electronic device to perform methods, such as neuromodulation therapy control methods, as described in the above examples, such as to treat one or more diseases or disorders. In an example, the instructions can include instructions to receive sensor data from one or more physiologic sensors and, based on the sensor data, control a therapy. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1-26. (canceled)

27. A method for controlling delivery of a neurostimulation therapy,
the method comprising:
receiving an acceleration signal from an accelerometer, wherein the accelerometer is configured for implantation in a submandibular region or cervical region of a patient;
determining a moving average of the acceleration signal over a first duration;
determining a moving standard deviation based on the moving average;
determining a first specified threshold based on the moving standard deviation;
identifying respiration phase transitions based on a relationship between the first specified threshold and the acceleration signal;
determining a respiration rate based on the identified respiration phase transitions;
determining a therapy withholding duration based on the respiration rate; and
providing neurostimulation therapy in coordination with an onset of an inspiration phase of a respiratory cycle of the patient, wherein the onset of the inspiration phase follows a first respiration phase transition of the identified respiration phase transitions by the therapy withholding duration.

28. The method of claim 27, wherein the first specified threshold is based on a product of the moving standard deviation and a specified sensitivity scalar.

29. The method of claim 27, wherein identifying the respiration phase transitions includes identifying respective times when a value of the acceleration signal meets or exceeds the first specified threshold.

30. The method of claim 29, wherein identifying the respiration phase transitions includes identifying respective times corresponding to an onset of exhalation.

31. The method of claim 27, comprising:
monitoring the respiration rate over time; and
in response to a detected change in the respiration rate, changing the therapy withholding duration.

32. The method of claim 27, wherein identifying the respiration phase transitions includes changing the first specified threshold to increase a likelihood that a value of the acceleration signal meets or exceeds the first specified threshold.

33. The method of claim 27, wherein determining the moving average of the acceleration signal includes identifying one or more blanking durations corresponding to one or more portions of the acceleration signal over the first duration, and determining the moving average without using the one or more portions of the acceleration signal that correspond to the one or more blanking durations.

34. The method of claim 33, comprising:
determining likely values of the acceleration signal for the one or more blanking durations; and
wherein determining the moving average of the acceleration signal includes using the determined likely values.

35. The method of claim 33, wherein determining the likely values of the acceleration signal includes using an interpolation function based on values of the acceleration signal outside of the one or more blanking durations.

36. A system comprising:
a first housing configured for implantation in a submandibular region or cervical region of a patient;
a first electrode lead coupled to the first housing and configured to be disposed in the submandibular region, wherein at least one electrode on the first electrode lead is configured to be disposed at or near a cranial nerve of the patient to provide a first neurostimulation therapy that is configured to treat a sleep disorder or breathing disorder of the patient;
an accelerometer configured to provide an acceleration signal that includes information about a respiration cycle of the patient; and
a processor circuit configured to:
receive the acceleration signal;
determine a moving average of the acceleration signal over a first duration;
determine a moving standard deviation based on the moving average;
determine a first specified threshold based on the moving standard deviation;
identify respiration phase transitions based on a relationship between the first specified threshold and the acceleration signal;
determine a respiration rate based on the identified respiration phase transitions;
determine a therapy withholding duration based on the respiration rate; and
provide a control signal to a signal generator circuit to provide the first neurostimulation therapy in coordination with an onset of an inspiration phase of a respiratory cycle of the patient, wherein the onset of the inspiration phase follows a first respiration phase transition, of the identified respiration phase transitions, by the therapy withholding duration.

37. The system of claim 36, wherein the processor circuit comprises a processor in communication with memory, the memory configured to store instructions that are executable by the processor and cause the processor circuit to perform one or more functions.

38. The system of claim 36, further comprising an antenna configured to exchange power and/or data between the system and an external device.

39. The system of claim 36, wherein the processor circuit is further configured to determine a number of the identified respiration phase transitions exceeds a respiration phase transition threshold within a specified time window, and, in response to determining the number of the identified respiration phase transitions exceeds the respiration phase transition threshold, provide a control signal to the signal generator circuit to provide neurostimulation therapy synchronous with respiration.

40. The system of claim 36, wherein the processor circuit is further configured to:
monitor the respiration rate over time; and
in response to detecting a change in the respiration rate, changing the therapy withholding duration.

41. The system of claim 36, wherein the first electrode lead is configured to be disposed at or near a first branch of a hypoglossal nerve of the patient.

42. The method of claim 27, wherein identifying respiration phase transitions comprises identifying a first set of respiration phase transitions, and further comprising:
determining a second moving average of the acceleration signal over a second duration;
determining a second moving standard deviation based on the second moving average;
determining a second specified threshold based on the second moving standard deviation;
identifying a second set of respiration phase transitions based on a relationship between the second specified threshold and the acceleration signal, the second set of respiration phase transitions being subsequent to the first set of respiration phase transitions;
determining an updated respiration rate based on the identified second set of respiration phase transitions;
determining an updated therapy withholding duration based on the updated respiration rate; and
providing updated neurostimulation therapy in coordination with the onset of the inspiration phase of a subsequent respiratory cycle of the patient, wherein the onset of the inspiration phase follows a first respiration phase transition of the identified second set of respiratory phase transitions by the updated therapy withholding duration.

43. The method of claim 27, wherein the therapy withholding duration comprises a specified percentage of the respiration rate.

44. The method of claim 27, further comprising determining a number of the identified respiration phase transitions exceeds a respiration phase transition threshold within a specified time window, and, in response to determining the number of the identified respiration phase transitions exceeds the respiration phase transition threshold, providing neuromodulation therapy synchronous with respiration.

45. The method claim 34, wherein determining the likely values of the acceleration signal includes using one or more a forecasting model, a regression model, or a pattern-matching model.

46. The method of claim 33, wherein identifying the one or more blanking durations comprises determining the one or more blanking durations based on an intensity or duration of the neurostimulation therapy.

47. The method of claim 33, wherein identifying the one or more blanking durations comprises determining the one or more blanking durations based on the determined respiration rate.
